(19) United States
(12) Patent Application Publication
(10) Pub. No.: US 2025/0339425 A1
(43) Pub. Date: Nov. 6, 2025

(54) Alpha9 and Alpha7 Nicotinic Acetylcholine Receptor Ligands

(71) Applicant: Northeastern University, Boston, MA (US)

(72) Inventors: Ganeshsingh THAKUR, Cambridge, MA (US); Hina Andleeb, Boston, MA (US)

(21) Appl. No.: 19/199,350

(22) Filed: May 5, 2025

Related U.S. Application Data
(60) Provisional application No. 63/642,643, filed on May 3, 2024.
(60) Provisional application No. 63/643,613, filed on May 7, 2024.

(51) Int. Cl. A61K 31/496; A61K 31/495

(52) U.S. Cl. CPC A61K 31/496; A61K 31/495

(57) ABSTRACT

Novel compounds which selectively target the nicotinic acetylcholine receptor (nAChR) α9, α10, and α7 subunits are provided. Substituted dialkylpiperazinium compounds, both with and without chiral switches, were identified as potent agonists exhibiting selectivity for human nAChRs containing α9 and α9α10 subunits over nAChRs containing α7 subunits. Chiral analogs demonstrated a preference for selectivity towards one receptor subtype over the other. The compounds can be used as α9- and α7-specific therapeutics for pain management. The compounds also exhibit inhibition of ATP-induced interleukin-1β release in THP-1 cells, indicating anti-inflammatory properties.

### CROSS REFERENCE TO RELATED APPLICATIONS

This application claims the priority of U.S. Provisional Application No. 63/642,643 filed 3 May 2024 and entitled “Alpha9 and Alpha7 Nicotinic Acetylcholine Receptor Ligands”, and U.S. Provisional Application No. 63/643,613 filed 7 May 2024 and entitled “Alpha9 and Alpha7 Nicotinic Acetylcholine Receptor Ligands”. Each of the aforementioned applications is hereby incorporated by reference in its entirety.

### BACKGROUND

Nicotinic acetylcholine receptors (nAChRs) are cation-selective, ligand-gated ion channels (LGICs) that mediate a diverse array of physiologic processes, including fast neurotransmission in the peripheral nervous systems (at the skeletal neuromuscular junction and in the autonomic nervous system), and modulation of synaptic function in the central nervous system, as well as immunomodulatory functions in peripheral tissues.^{1, 2}

Functional nAChRs as LGIC result from the assembly of five either identical or different subunits, giving rise to homomeric or heteromeric pentamers, respectively.³ Neuronal nAChRs are formed from amongst nine identified α (α2 to α10) and three β (β2 to β4) subunits.⁴ It has been proposed that nAChR function can be modulated to treat various nervous system disorders, such as Alzheimer's disease, schizophrenia, depression, attention deficit hyperactivity disorder (ADHD), and tobacco addiction,^{5, 6} as well as chronic pain⁷ Parkinson's disease⁸ and hearing disorders⁹. Much of the work regarding nAChRs as therapeutic targets has focused on the subtypes expressed at high levels in the brain, heteromeric receptors containing α4 and β2 subunits and homomeric α7-containing receptors. However, the identification of nAChR expression in a variety of immune cells has provided evidence for a cholinergic anti-inflammatory system (CAS)^{10, 11} that modulates inflammatory disease and neuropathic pain. This discovery has promoted a new direction for the development of pain therapeutics. In addition to their canonical ionotropic functions, non-canonical, flux-independent and exclusively metabotropic functions have been proposed for nAChRs and other ligand-gated ion channels. The function of nAChRs in the CAS may rely more on metabotropic than ionotropic signaling¹²⁻¹⁴ and the receptor subunits most strongly implicated as targets are α7 and the less-well-understood α9 and α9α10 receptor subunits^{15, 16}, which until recently have only been associated with auditory function.^{17, 18} The α9 subunits are known to combine with α10 subunits to form α9α10 nAChRs with kinetic properties slightly different from homomeric α9 nAChRs, with a likely (α9)₂(α10)₃ stoichiometry.¹⁹

The heteromeric nAChRs on hair cells contain α9 and α10 subunits and have several distinguishing characteristics.²⁰ The α9α10 receptor features antagonism by nicotine, which, as noted above, typically activates nAChRs, and potent block by strychnine and bicuculline, which are also antagonists of glycine and GABA receptors, respectively.²¹ The α9α10 nAChRs are among the most calcium-permeable LGICs known²² although their endogenous ion channel activity has only been recorded in cochlear and vestibular hair cells. However, expression of α9α10 has been described in dorsal root ganglion neurons^{23, 24} lymphocytes, skin keratinocytes, and the pars tuberalis of the pituitary gland.²⁵ This widespread distribution of α9* nAChRs may be associated with diverse physiological roles for these receptors in neuronal, sensory, metabolic, and immune tissues¹⁸. The α9 and α10 subunits share homology with other nAChRs, yet are structurally and pharmacologically distinct, having the lowest degree of sequence similarity with other nAChRs, making them a promising target for developing selective drugs. In addition to potentially modulating CAS, compounds that target α9 and α9α10 may be useful for treating various hearing disorders, such as noise-induced hearing loss or the debilitating disorders, vertigo, or tinnitus.¹⁸

It has recently been shown that compounds previously identified as silent agonists of α7, with potential metabotropic activity, could function as potent α9 agonists or antagonists.²⁶ It was confirmed that one of the α9 agonists was an effective inhibitor of the ATP-induced maturation and release of the pro-inflammatory cytokine interleukin (IL)-1β in a cell-based assay.²⁷

There is a need to develop specific modulators of nAChRs containing α7, α9, and α10 subunits so as to treat and prevent conditions related to their physiological roles. In particular, there is an urgent need for non-opioid treatments for chronic and neuropathic pain to provide effective alternatives amidst the escalating opioid crisis.

### SUMMARY

The present invention discloses novel compounds designed to selectively target the α9, α10, and α7 nicotinic acetylcholine receptor (nAChR) subunit, which plays a crucial role in pain regulation, inflammation, and inner ear functions. Specifically, the invention identifies substituted dialkylpiperazinium compounds, both with and without chiral switches, as potent agonists exhibiting selectivity for human α9 and α9α10 nAChRs over α7 nAChRs. Chiral analogs demonstrate a preference for selectivity towards one receptor subtype over the other. Notably, compounds II, Vf and Vg demonstrate significant potency and selectivity. Compound II functions as a full agonist at α9 nAChRs, displaying a remarkable 340-fold selectivity over α7. Additionally, it exhibits inhibition of ATP-induced interleukin-1β release in THP-1 cells, indicating potential anti-inflammatory properties. Importantly, the analgesic efficacy of these compounds remains unaffected in α7 knockout mice, suggesting mediation through α9* nAChRs. These findings present a promising avenue for the development of α9* and α7-specific therapeutics tailored for pain management. As used herein, α9* represents α9 homomeric receptors as well as α9α10 heteromers considered as a group.

The technology can be further summarized with the following list of features.

1. A compound of Formula I, wherein the compound binds to a nicotinic acetylcholine receptor comprising an alpha9, alpha10 and/or alpha7 subunit:

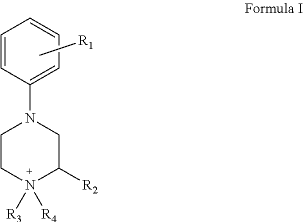

wherein R₁ is selected from the group consisting of cyano or —R₅—R₆; wherein R₅ is —CONH—, —NHCO—, oxazole, or oxadiazole; wherein R₆ is C1-C6 alkyl, C1-C6 haloalkyl or dihaloalkyl, halopyridyl, cyanopyridyl, C1-C6 alkylpyridyl, optionally further halo substituted, 1-pentynyl, ethenylcyclopropyl, methenylcyclobutyl, or 2-quinolyl;
wherein R2 is hydrogen or C1-C6 alkyl; wherein R₂ can be bound to any carbon on the piperazine ring and with either R or S stereochemistry;
wherein R3 and R4 are independently C1-C6 alkyl or hydrogen, with the proviso that if one of R3 and R4 is hydrogen, the other is not hydrogen, and optionally wherein R3 and R4 are fused to form a 4-, 5-, 6-, or 7-membered ring, saturated or unsaturated, optionally containing 1 or 2 heteroatoms selected from N, O, and S, and optionally substituted; and
wherein all substitutions on 6-membered rings can be ortho, meta, or para.
   2. The compound of feature 1, wherein the compound is an agonist, partial agonist, or silent agonist of said nicotinic acetylcholine receptor.
   3. The compound of feature 1, wherein the compound is an antagonist of said nicotinic acetylcholine receptor.
   4. The compound of feature 1, wherein the compound is selected from group consisting of the following compounds:

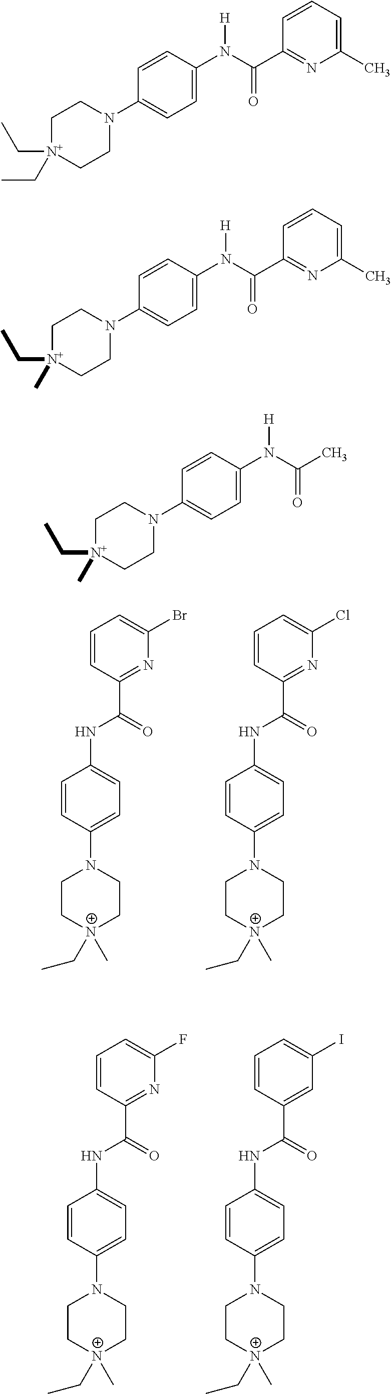

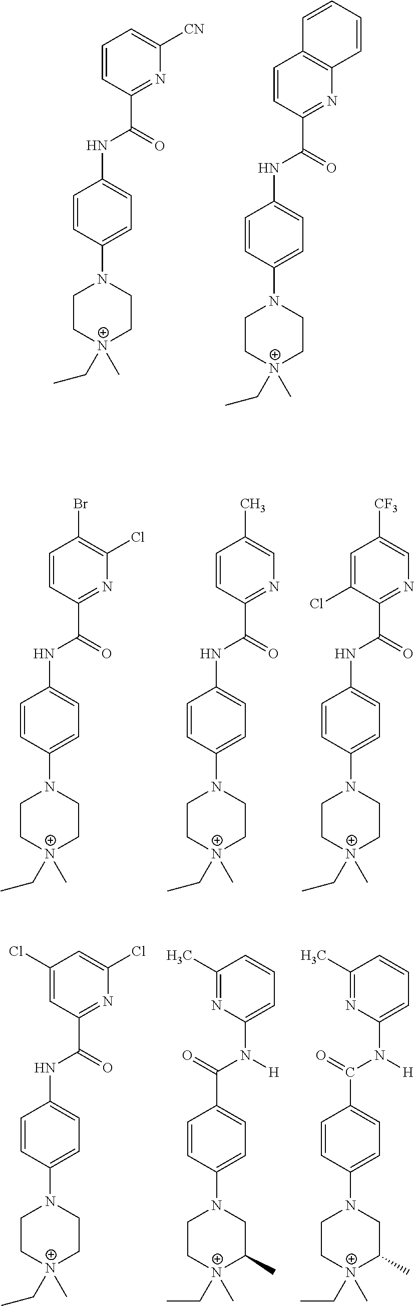

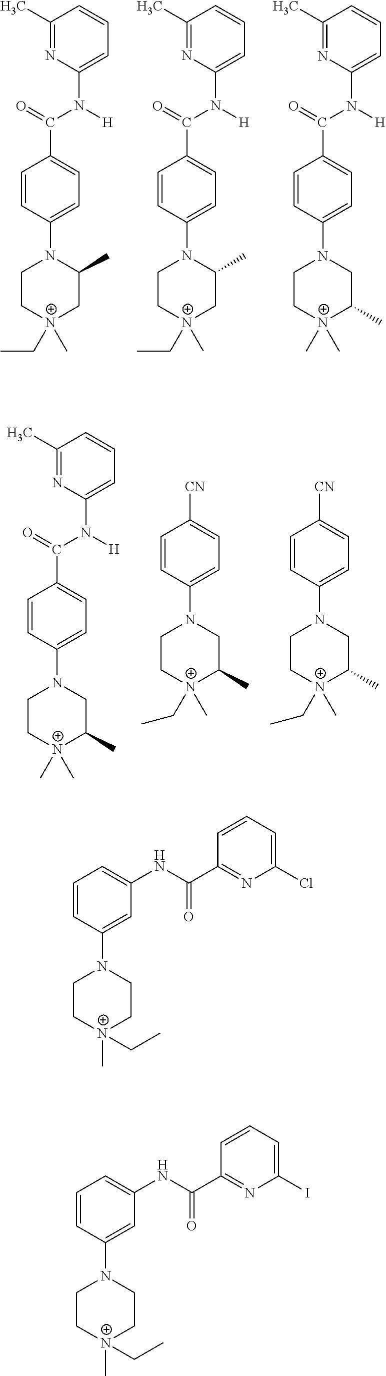

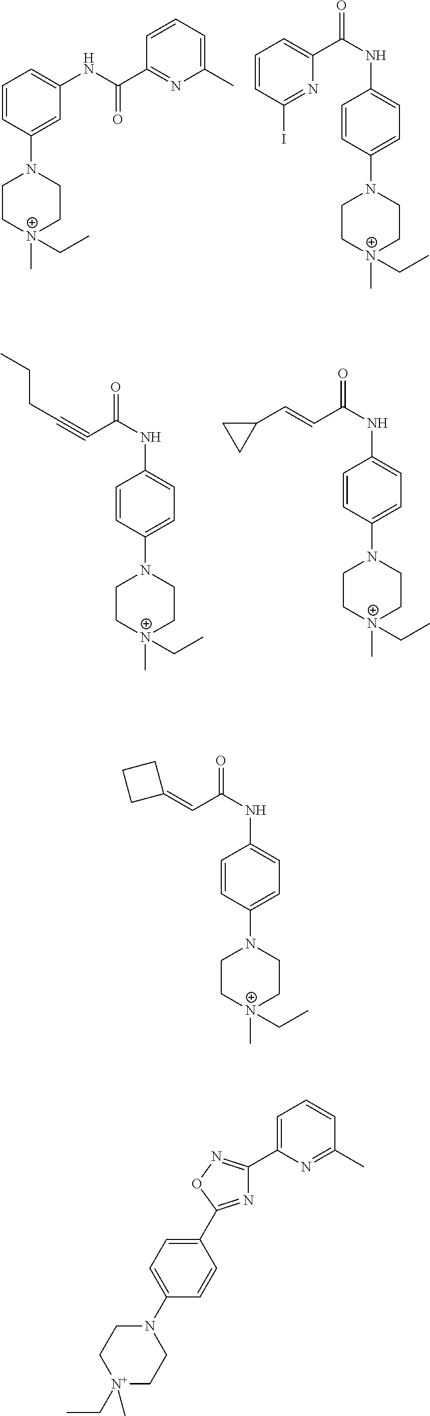 [SMILES: CC[N+]1(C)CCN(c2cccc(NC(=O)c3cccc(C)n3)c2)CC1 ; CC[N+]1(C)CCN(c2ccc(NC(=O)c3cccc(I)n3)cc2)CC1 ; CCCC#CC(=O)Nc1ccc(N2CC[N+](C)(CC)CC2)cc1 ; CC[N+]1(C)CCN(c2ccc(NC(=O)/C=C/C3CC3)cc2)CC1 ; CC[N+]1(C)CCN(c2ccc(NC(=O)C=C3CCC3)cc2)CC1 ; CC[N+]1(C)CCN(c2ccc(-c3nc(-c4cccc(C)n4)no3)cc2)CC1]

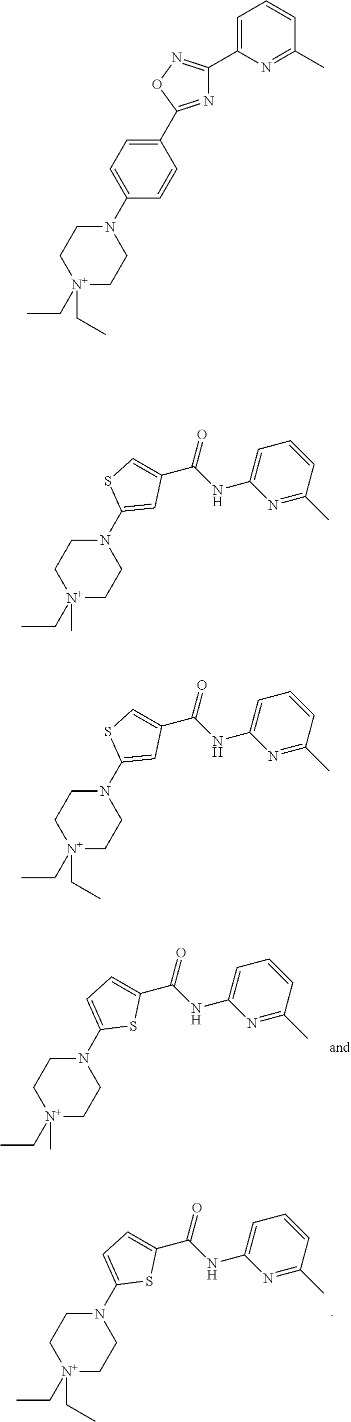

5. The compound of any of the preceding features, wherein the compound is at least 70%, at least 80%, at least 90%, at least 95%, at least 98%, or at least 99% enantiomerically pure with respect to one or more chiral sites.
6. The compound of any of the preceding features, wherein the compound is peripherally active and does not substantially cross the blood-brain barrier.
7. The compound of any of features 1-5, wherein the compound is at least partially uncharged at physiological pH and is capable of crossing the blood-brain barrier.
8. The compound of any of the preceding features, wherein the compound is present as a pharmaceutically acceptable salt, such as a halide, or a salt formed with an acid, such as a hydrochloride.
9. The compound of any of the preceding features, wherein the compound has a dissociation constant of less than about 1 μM, or less than about 300 nm, or less than about 200 nM, or less than about 100 nM, for a form of the nicotinic acetylcholine receptor comprising one or more alpha7, alpha9, and/or alpha10 subunits.
10. The compound of any of the preceding features, wherein the compound has a binding selectivity for alpha9-containing forms and/or alpha9-alpha10-containing forms of the nicotinic acetylcholine receptor over alpha7-containing forms of the nicotinic acetylcholine receptor of at least 50, at least 100, at least 150, at least 200, or at least 250.
11. The compound of any of the preceding features, wherein the compound decreases pain and/or inflammation when administered to a mammal at an effective dose.
12. A pharmaceutical composition comprising the compound of any of the preceding features and at least one excipient.
13. The pharmaceutical composition of feature 12, further comprising one or more additional active agents.
14. The pharmaceutical composition of feature 13, wherein the one or more additional agents comprise an agent for treatment of pain, inflammation, or cancer.
15. A method to aid in treating, or preventing or alleviating to any degree, a disorder related to a nicotinic acetylcholine receptor comprising an alpha9, alpha10 and/or alpha7 subunit, the method comprising administering to a mammalian subject in need thereof an effective amount of the compound of any of features 1-11 or the pharmaceutical composition of any of features 12-14.
16. The method of feature 15, wherein the disorder is selected from the group consisting of sensory and auditory disorders; hearing loss (including noise-induced, age-related, or ototoxic); tinnitus; pain; inflammation; neuropathic pain; chronic pain (including inflammatory, musculoskeletal, cancer-induced); visceral pain (including interstitial cystitis and irritable bowel syndrome); neurodegenerative disorders; neurological disorders; multiple sclerosis; Parkinson's disease; peripheral neuropathy; autoimmune disorders; rheumatoid arthritis; Inflammatory bowel disease (including Crohn's disease, ulcerative colitis); cancer (including cancer chemotherapy and pain related to oral, bone, or visceral cancers; chemotherapy-induced hearing loss or neuropathy; preventive care related to platinum-based or taxane-based chemotherapies; and cancer immunomodulation.
17. The method of feature 15, wherein the disorder is selected from the group consisting of pain, chronic pain, neuropathic pain, inflammation, inflammatory pain, neuroinflammation, tinnitus, and an inner ear disorder.

### BRIEF DESCRIPTION OF THE DRAWINGS

FIG. 1A shows an embodiment of a genus of compounds according to the present technology. FIGS. 1B-1E show optional components for each of Sites I-IV of the embodiment shown in FIG. 1A.

FIGS. 2A-2C show the concentration dependence of whole cell ionic currents obtained from Xenopus oocytes expressing the indicated subunits of nAChR, for each of Compound I (FIG. 2A), Compound II (FIG. 2B), and Compound III (FIG. 2C). The results are normalized to the current obtained with a maximal dose of Ach alone.

FIG. 3A shows a schematic representation of the interactions among signaling pathways inhibited by nAChR in a mononuclear phagocytic cell. FIG. 3B shows a summary of the results obtained for IL-1β release by THP-1 cells using the indicated conditions (see Example 9).

FIG. 4 shows the stability of the indicated compounds in mouse plasma over time.

FIGS. 5A-5D show results from administration of complete Freunds adjuvant (CFA) in a mouse paw pad inflammation assay using the indicated compounds.

FIGS. 6A-6D show the concentration dependence of whole cell ionic currents obtained from Xenopus oocytes expressing the indicated subunits of nAChR, for the indicated compounds. The results are normalized to the current obtained with a maximal dose of Ach alone.

FIG. 7 shows the concentration dependence of whole cell ionic currents obtained from Xenopus oocytes expressing the α9 subunit of nAChR, for the indicated compounds. The results are normalized to the current obtained with a maximal dose of Ach alone.

FIGS. 8A-8H show the concentration dependence of whole cell ionic currents obtained from Xenopus oocytes expressing the indicated subunits of nAChR, for the indicated compounds. The results are normalized to the current obtained with a maximal dose of Ach alone.

FIGS. 9A-9D show crystal structures displayed in ball and stick model for enantiomers (2R)PA-diMPP (FIG. 9A) and (2S)PA-diMPP (FIG. 9C) showing the orientation of the methyl and C—C bonds in the flipped chair form in the case of FIG. 9C. FIGS. 9B and 9D are the equivalents of 9A and 9C, respectively. The large separate ball in 9A and 9C is the iodide ion.

FIGS. 10A-10B show evaluation of methyl modifications of PA-EMPP based on ionic current measurements in Xenopus oocytes. After the acquisition of initial 60 μM ACh control responses, 30 μM applications were made of the parent compound or the stereoisomer when methyl groups were added to the piperazine ring at either the 2 or the 3 position. The peak current responses were normalized to the average of the two initial responses to ACh.

FIGS. 11A-11C show receptor activation responses to the indicated PA-EMPPs. Concentration-response studies for (2R)r/sPA-EMPP (4) and (2S)r/sPA-EMPP (5) on cells expressing α7, α9, or α9α10 nAChR as indicated. Peak current for 8 cells (±SEM) was measured across a range of compound concentrations, and each response was normalized to a preceding ACh control response. Reponses relative to 60 μM controls were then scaled to reflect the difference between ACh controls and ACh maximum responses.

FIGS. 12A-12C show receptor activation responses for the indicated diMPPs. Concentration-response studies for chiral diMPP compounds (2R)PA-diMPP (8) and (2S)PA-diMPP (9) on cells expressing α7, α9, or α9α10 nAChR as indicated. Peak current for 8 cells (±SEM) was measured across a range of compound concentrations, and each response was normalized to a preceding ACh control response. Reponses relative to 60 μM controls were then scaled to reflect the difference between ACh controls and ACh maximum responses.

FIGS. 13A-13B show inhibition concentration-response studies for chiral diMPP compounds (2R)PA-diMPP (8) and (2S)PA-diMPP (9), identified as α9 and α9α10 antagonists. Peak currents for 6-8 cells (±SEM) were measured across a range of compound concentrations co-applied with 60 μM ACh and normalized to preceding responses to ACh alone.

FIGS. 14A-14B show evaluation of 2M isomers of pCN-EMPP with concentration-response studies of α7 and α9α10 receptors as indicated. Peak currents were obtained from 5 cells (±SEM).

FIG. 15 shows the impact of (2R)r/sPA-EMPP and (2S)r/sPA-EMPP on the BzATP-mediated release of interleukin (IL)-113 by human monocytic THP-1 cells. Monocytic THP-1 cells were primed with lipopolysaccharide (LPS; 1 μg/ml, 5 hours). Thereafter, the P2X7 receptor agonist BzATP (100 μM) was added for another 40 minutes to trigger IL-1β release. The BzATP-induced release of IL-1β was investigated in the presence and absence of acetylcholine (ACh, 10 μM), (2R)r/sPA-EMPP or (2S)r/sPA-EMPP. The concentration of IL-1β released in response to BzATP was calculated by subtracting the IL-1β concentrations measured in supernatants of cells treated with LPS alone. The IL-1β concentrations obtained after stimulation with BzATP+solvent were set to 100% and all other values were calculated accordingly. Data are presented as individual data points, the bar represents the median, and whiskers encompass the 25^{th} to 75^{th} percentile. *p≤0.05, different from LPS-primed cells stimulated with BzATP alone. Friedman test followed by the Wilcoxon signed-rank test.

FIGS. 16A-16B show the impact of (2R)PA-diMPP and (2S)PA-diMPP on the BzATP-mediated release of interleukin (IL)-1β by human monocytic THP-1 cells. Conditions were as described for FIG. 15.

FIG. 17 shows the impact of (2R)r/s-pCN-EMPP and (2S)r/s-pCN-EMPP on the BzATP-mediated release of interleukin (IL)-1β by human monocytic THP-1 cells. Conditions were as described for FIG. 15.

### DETAILED DESCRIPTION

The present technology provides novel compounds targeting the α9 nicotinic acetylcholine receptor (nAChR) subunit, which is crucial for pain regulation, inflammation, and inner ear functions. The novel compounds include substituted carbamoyl/amido/heteroaryl dialkylpiperazinium iodides, which act as potent agonists selective for human nAChR containing α9 and α9α10 subunits over nAChR containing α7 subunits.

An earlier generation of compounds having binding selectivity for α9 are described in U.S. Pat. No. 11,884,629 B2, which is hereby incorporated by reference. Some of the present compounds reverse the amide linkage to create reverse amide analogs, thus examining the effect of altering the distance of the hydrogen bond donor to the binding site residues. The phenylpiperazine scaffold was optimized for selective binding to α9 containing receptors, and the resulting compounds offer treatment methods related to the roles of nAChRs in auditory, antinociceptive, and anti-inflammatory processes.

The present invention addresses the long-standing challenge of higher effective drugs for chronic, inflammatory and neuropathic pain. In spite of the rapid progress achieved in the field of chronic pain, its effective management in clinical settings remains highly challenging. Strategies for treating pain have shown limited advancement over the course of decades, largely relying on opioids as the primary prescribed medications for chronic pain relief. However, these prior solutions with opioids have fallen short in several key aspects, such as adverse side effects with detrimental outcome. These shortcomings have necessitated the development of a more effective and innovative solution, which is the focus of the present invention.

Previous attempts have been made to address these medical needs via the alpha 7 receptor. However, the only effective agents described for the treatment of pain and inflammation via nAChR have been with p-CN-diEPP and amide containing dialkylpiperazinium salts. Here, novel solutions are presented based on explorations of highly selective and potent alpha 9 agonists and their role in pain and inflammation.

The invention includes certain compounds that have activity as selective nAChR α9-selective agonists or partial agonists, and may have activity as selective nAChR α7 antagonists, or have little or no effect on α7. The compounds fall within the general formula as follows:

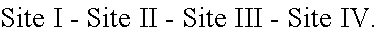

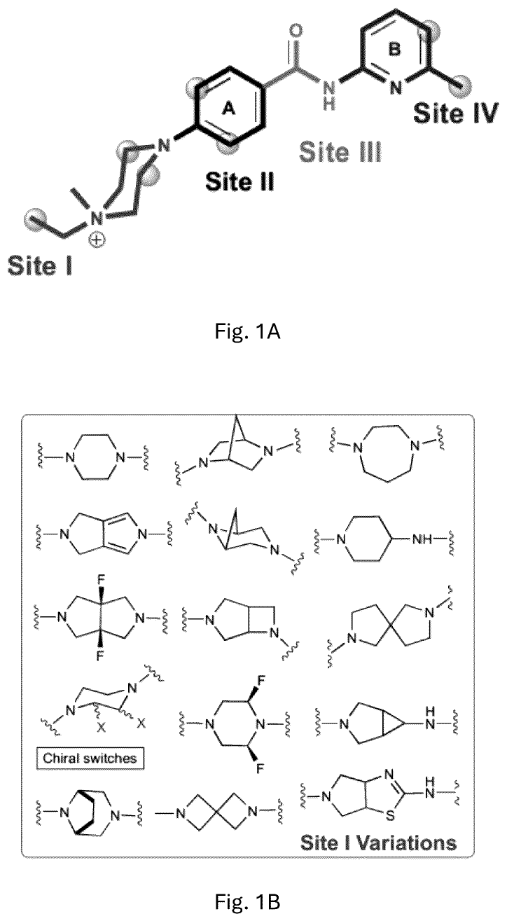

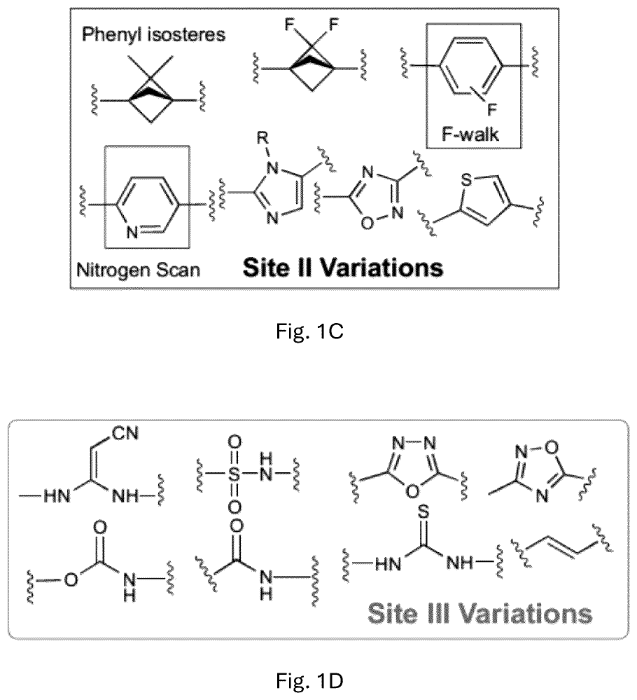

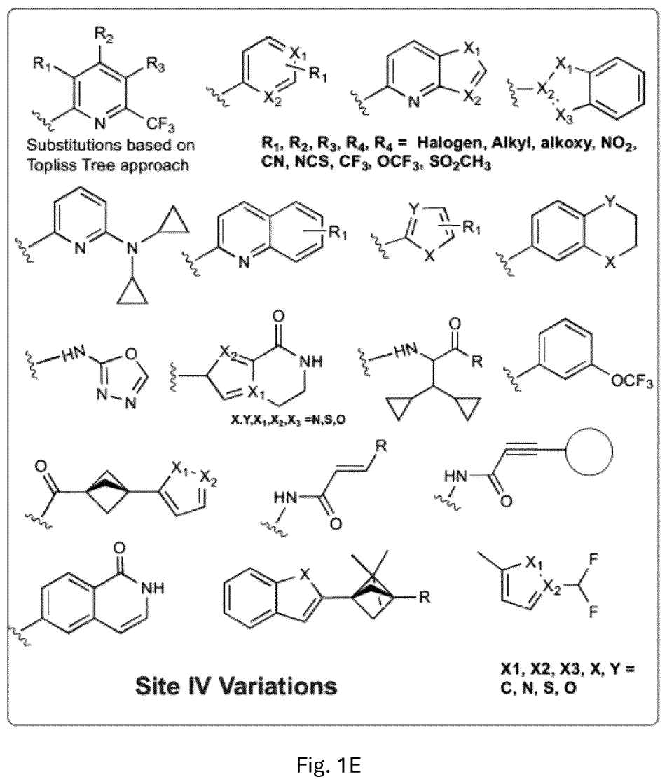

Preferred components of each site and their relationship are depicted in FIGS. 1A-1E. The preferred components of Sites I, II, III, and IV as shown in FIGS. 1B-1E can be combined as any selection for Site I, covalently linked to any selection for Site II, covalently linked to any selection for Site III, covalently linked to any selection from Site IV. Where appropriate, such as on ring structures and alkyls, including where appropriate at ortho, meta, or para positions, additional substitutions can be made. Possible substituents include the following: hydrogen, hydroxy, sulfoxy, halo, acyl, acyloxy, alkyl, heteroalkyl, alkenyl, heteroalkenyl, alkynyl, heteroalkynyl, alkoxy, cycloalkyl, heterocycloalkyl, aryl, arylalkyl, arylhalo, arylhydroxy, arylcyano, aryltrifluoromethyl, aryltrifluoromethoxy, arylnitro, aryltrifluoromethoxy, arylnitro, and arylether, arylester, arylsulfonyl, arylsulfinyl, arylsulfonamidyl, arylsulfonate, arylsulfoxyl, arylphosphate ester, arylcarbonyl, arylcarboxylate, arylcarbamate, arylamine, arylimide, heteroaryl, heteroarylalkyl, heteroarylhalo, heteroarylhydroxy, heteroarylcyano, heteroaryltrifluoromethyl, aryltrifluoromethoxy, arylnitro, heteroaryltrifluoromethoxy, heteroarylnitro, and heteroarylether, heteroarylester, heteroarylsulfonyl, heteroarylsulfinyl, heteroarylsulfonamidyl, heteroarylsulfonate, heteroarylsulfoxyl, heteroarylphosphate ester, heteroarylcarbonyl, heteroarylcarboxylate, heteroarylcarbamate, heteroarylamine, heteroarylimide, quinidine, morpholine, and any ring structure is optionally substituted with any of the substituents described herein, with the proviso that any two adjacent substituents can come together to form a carbocyclic or heterocyclic ring system. A hydrocarbon or heterocyclic ring system can be phenyl, thienyl, furanyl, pyrimidinyl, oxazoyl, thiazolyl, pyridyl, naphthyl, quinolinyl, indolyl, benzothiophenyl, benzofuranyl, pyrrolyl, imidazolyl, pyrazole, triazolyl, isoxazolyl, pyridazinyl, pyzazinyl, pyrimidinyl, oxadiazolyl, benzimidazolyl, or triazinyl. A heterocyclic ring system may contain one or more heteroatoms selected from the group consisting of oxygen, sulfur, nitrogen, and combinations thereof.

The inventors have synthesized and characterized novel 1,1-dialkyl-4-(4-substituted arylcarboxamido/reverse-amido)phenyl)piperazin-1-ium iodides with (see Compounds IVa-j below) and without chiral switches (see Compounds I-III and Va-j below) and explored their agonist activity and selectivity for targeting α9 and α9α10 over α7 nAChR.

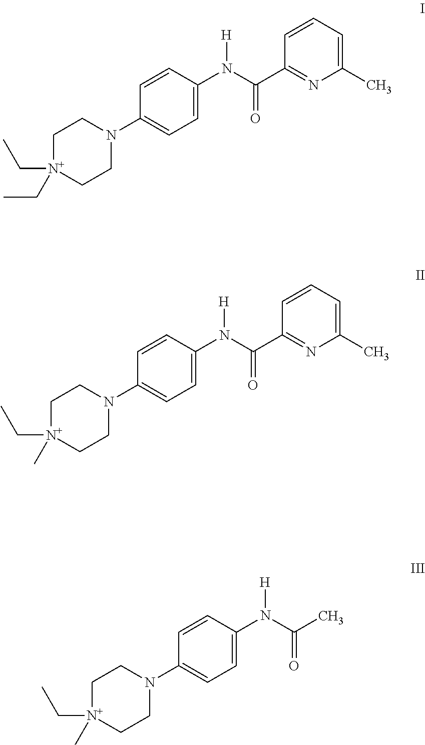

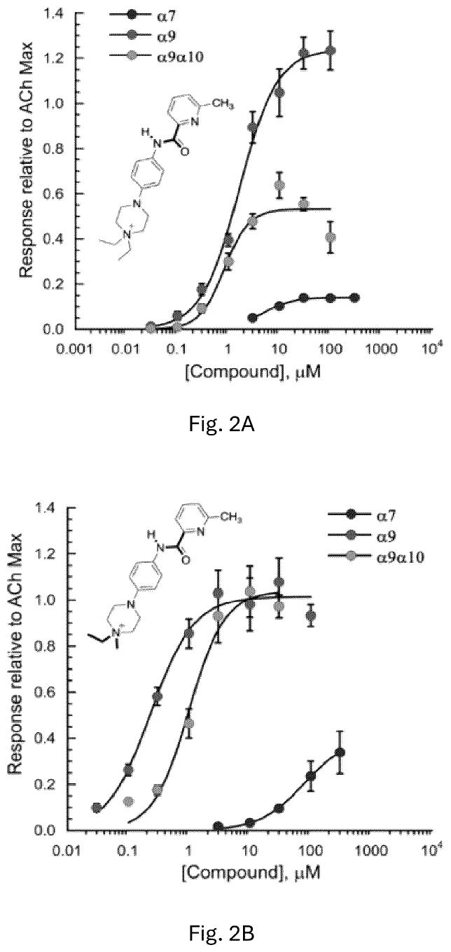

Xenopus oocytes were injected with RNAs to produce functional nAChRs, which were characterized by two-electrode voltage-clamp measurements. FIGS. 2A-2C present concentration-response curves (CRCs) for the new compounds tested with human α9, and α9 α10 vs α7 receptors; Table 1 presents the values for Iₘₐₓ and EC₅₀ with these receptors for new 1,1-dialkyl-4-substituted phenylpiperazinium iodides with α9-containing and α7 nAChR.

The results indicate that compounds I-III functioned as α9-selective full agonists, with higher selectivity over α7 receptors. Notably, compounds I and III displayed robust partial agonism for α9α10 (FIGS. 2A-2C, Table 1). When the picolinyl group in compound II was substituted with a methyl group, as seen in compound III, there is a marked shift in receptor selectivity favoring α9* over α7 nAChR. Additionally, the agonist activity of the compound transitioned from full to partial agonism on α9* with reduced potency (FIG. 2C, Table 1). These observations suggest that smaller alkyl groups, such as methyl, are potentially not optimal for the α9* binding pocket, given the greater spatial availability in the extended binding region. Among the reverse amide analogs, compound III emerged as the most potent α9 full agonist, exhibiting a potency of 230 nM-remarkably, the highest across all compound series. This represents a 113-fold increase in potency relative to ACh. Importantly, reverse amide II is twice as potent as its normal amide counterpart, 3f. Overall compound II was found to be 340-fold selective for α9 over α7 nAChR.

TABLE 1
Iₘₐₓ and EC₅₀ values of normal amide 3f vs reverse amide analogs (I-III) on
α9-containing and α7 nAChR.

| Compd. | | α9 | | α9α10 | | α7 | |
|---|---|---|---|---|---|---|---|
| No. | Structures | Iₘₐₓ^{a} | EC₅₀^{b} | Iₘₐₓ | EC₅₀ | Iₘₐₓ | EC₅₀ |
| 3f (see ref) | 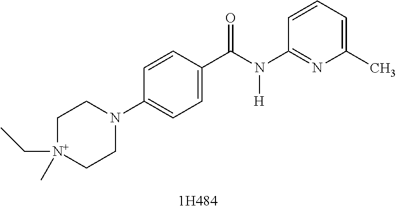 [SMILES: CC[N+]1(C)CCN(c2ccc(C(=O)Nc3cccc(C)n3)cc2)CC1] | 1.19 ± 0.03 | 0.51 ± 0.07 | 0.82 ± 0.04 | 0.48 ± 0.09 | 0.40 ± 0.02 | 6.1 ± 1.1 |
| | Normal amide | | | | | | |
| | U.S. Pat. (pat. | | | | | | |
| | No. U.S. = | | | | | | |
| | 11,884,629, B2) | | | | | | |
| I | 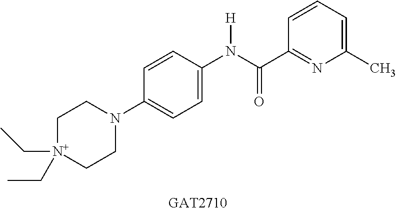 [SMILES: CC[N+]1(CC)CCN(c2ccc(NC(=O)c3cccc(C)n3)cc2)CC1] | 1.24 ± 0.04 | 1.63 ± 0.20 | 0.53 ± 0.04 | 0.83 ± 0.25 | 0.14 ± 0.004 | 4.68 ± 0.54 |
| | Reverse amide | | | | | | |
| II | 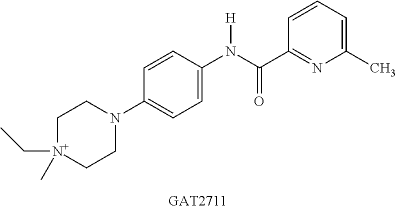 [SMILES: CC[N+]1(C)CCN(c2ccc(NC(=O)c3cccc(C)n3)cc2)CC1] | 1.01 ± 0.03 | 0.23 ± 0.034 | 1.04 ± 0.07 | 0.99 ± 0.21 | 0.41 ± 0.025 | 78.34 ± 11.32 |
| | Reverse amide | | | | | | |
| III | 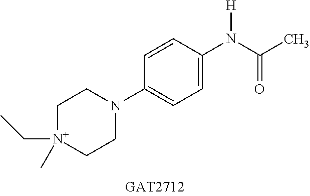 [SMILES: CC[N+]1(C)CCN(c2ccc(NC(C)=O)cc2)CC1] | 0.75 ± 0.04 | 2.27 ± 0.49 | 0.64 ± 0.02 | 3.30 ± 0.29 | — | — |
| | Reverse amide | | | | | | |
| | ACh | 1.00 ± 0.04 | 26 ± 4 | 1.00 ± 0.04 | 30 ± 10 | 0.97 ± 0.04 | 21 ± 3 |

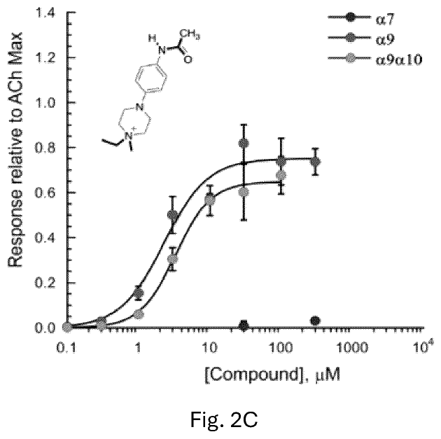

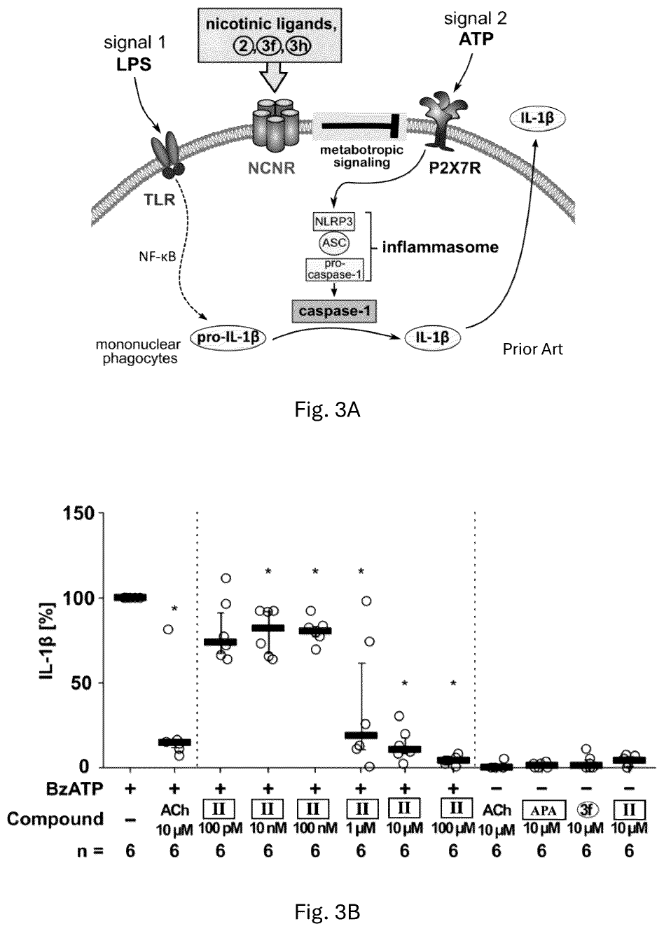

Previous studies have demonstrated that stimulation of nAChRs containing α7 and/or α9/α10 subunits potently inhibits the ATP-induced inflammasome-dependent cleavage and release of the pro-inflammatory cytokine IL-1β by monocytic cells. 34, 35 See FIG. 3A for schematic illustration of the underlying mechanisms. The new compounds were tested for the ability to down-regulate the ATP-mediated release of 10 IL-1β by human monocytic THP-1 cells. The cells were primed with lipopolysaccharide (LPS; 1 μg/ml) for 5 h and stimulated for another 40 min with the P2X7 receptor agonist (2′(3′)-O-(4-benzoyl-benzoyl) ATP (BzATP, 100 μM) in the presence or the absence of ACh (10 μM) and different concentrations of compounds APA, 3f, or II (FIG. 3B). IL-1β concentrations were measured in cell culture supernatants by ELISA. Untreated cells (IL-1β=1 μg/ml) and cells primed with LPS (IL-1β=7 μg/ml, n=24) released low amounts of IL-1β, while priming of cells with LPS followed by stimulation with BzATP resulted in elevated IL-13 levels (IL-1β=78 μg/ml, n=24; FIG. 3B). Compounds APA, 3f, and II significantly and dose-dependently inhibited (IC₅₀ values APA=14 μM; 3f=9 μM; II=0.5 μM) the BzATP-induced release of IL-1β, similar to ACh^{29, 34, 35} which was included as a positive control (FIG. 3B). Compounds 3f and II appeared to be more effective compared to compound II (FIG. 3B). In the absence of BzATP, neither ACh nor compounds APA, 3f, or II induced the release of IL-1β by THP-1 cells that were primed with LPS (FIG. 3B). In none of the experimental settings was cell death increased, as measured by the lactate dehydrogenase activity in cell culture supernatants (data not shown).

These in vitro experiments suggest that compounds APA-diEPP, 3f, and II hold therapeutic promise for alleviating inflammation resulting from cellular damage of diverse origins, as well as for pain management. Additionally, nAChR stimulation in mononuclear phagocytes activates a broad array of anti-inflammatory signaling pathways.³⁶ The nAChR-mediated control of ATP signaling is only one of them. Of these, the control of ATP signaling via α9* nAChRs appears most pertinent in the modulation of ATP-induced IL-13 release, although further experimental validation is required to substantiate this mechanistic inference.

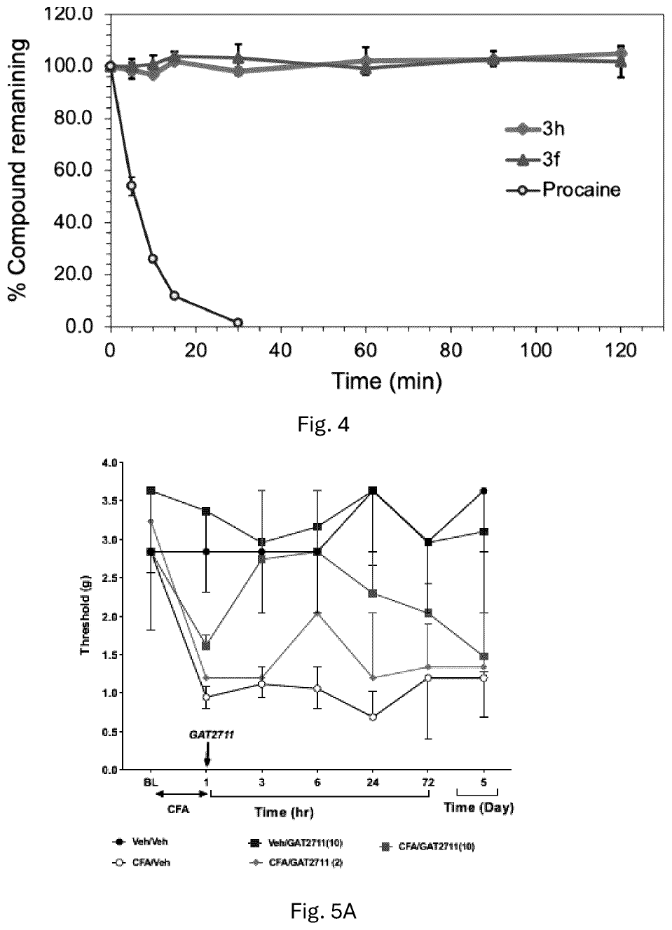

Compounds II and 3f exhibited high aqueous solubility (>1000 μM;) and were found stable in pooled murine plasma (FIG. 4). The plasma stability was observed using plasma from BALB/c mice. Half-life of procaine in plasma was found to be less than 5 min, but both compounds II and 3f were stable (>90%) up to 1 h incubation in murine plasma at 37° C. (FIG. 4). In vitro metabolic stability studies of both compounds were performed in pooled human liver microsomes and rat hepatocytes. Both compounds showed low clearance, and extrapolated hepatic extraction ratios are <0.12 in both microsomal- and hepatocyte-based assays. Plasma protein binding of II and 3f was 54 and 32%, respectively.

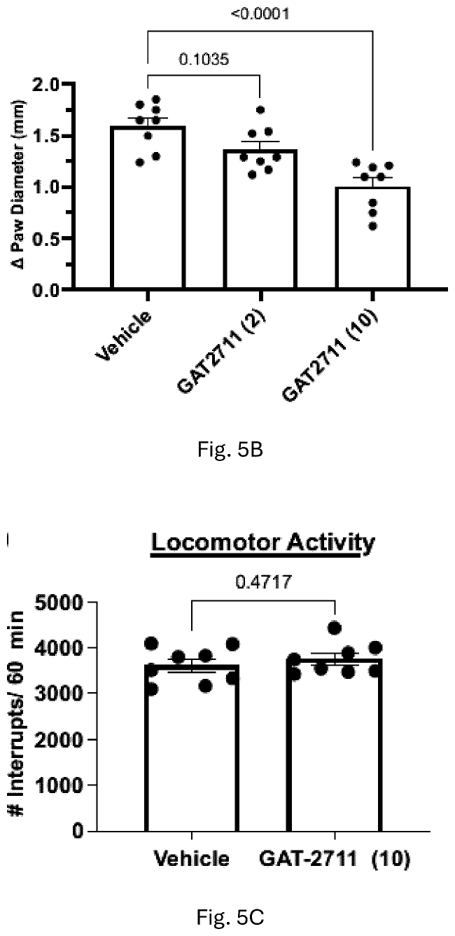

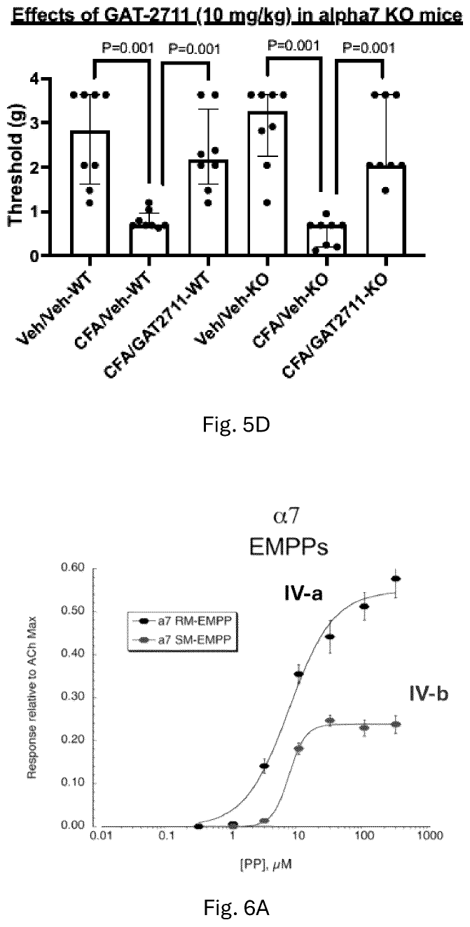

Animals treated with complete Freund's adjuvant (CFA) showed greatly reduced mechanical pain thresholds compared to vehicle-treated controls (P=0.001). Mechanical thresholds were elevated by treatments with II (FIGS. 5A-5D). Note that these data were not well fit to normal distributions, especially under control conditions where the majority of responses were the same high threshold value of 3.63 g. Therefore, the data in FIGS. 5A-5D are the mean±SD scores for each condition. Statistical analysis was conducted using a Kruskal-Wallis test followed by Mann-Whitney rank sum test at each time point following II treatment. There were significant effects for the dose of 10 mg/kg body weight (bw) tested at the 1 and 3 h time points (P=0.002 and 0.002), and for 2 and 10 mg/kg bw doses at the 6 h (P=0.01 and 0.002) and the 24 h time point (P=0.024 and 0.045) and for 10 mg/kg dose at the 5 days time point (P=0.002) with no significant effects at the 72 h time point. In sham-treated mice 10 mg/kg II did not alter von Frey responses. In addition, Compound II significantly reduced paw edema (F(2,21)=13.65; P=0.0002, Student's t test; FIG. 5B), with mice treated with 10 mg/kg dose differing from the vehicle group (P<0.0001). At that dose of 10 mg/kg, Compound II did not significantly alter locomotor activity of mice compared to vehicle-treated animals (P=0.4717) (FIG. 5C) as assessed by Student's t test.

In a separate experiment (FIG. 5D), CFA-treated WT and α7 nAChR KO mice were treated with Compound II (10 mg/kg bw) or vehicle and evaluated their mechanical hypersensitivity 3 days after CFA. The experiments indicated that the antinociceptive effect of Compound II was independent of nAChRs since the effects of 10 mg/kg bw II were essentially the same in both WT and α7 nAChR KO animals (Kruskal-Wallis test followed by Mann-Whitney rank sum test; Supplementary Table 1).

The present results have shown that Compound II has 340-fold higher selectivity for α9 over α7 nAChR, indicating that Compound II offers a novel approach to managing inflammatory pain or neuropathic pain. The retention of full analgesic activity of II in α7 nAChR knockout animals strongly implicates an α9* nAChR-dependent mechanism for Compound II. Further, the present cell-based assays indicate that α9 agonists and partial agonists can mediate α9*-dependent anti-inflammatory activity.

The novel agonists exhibited an extended duration of action. In contrast to previously²⁹ reported α9 nAChR agonists such as pCN-diEPP, which demonstrated efficacy in ameliorating CFA-induced pain like behaviors for a period ranging from 3-6 hours, Compounds II and 3f showed a significantly prolonged effective duration of approximately 72 hours. This extended analgesic effect is unlikely attributable to a potential depot effect linked to their quaternary ammonium groups, as this is a common feature shared among these compounds. Furthermore, the activation of α9*nAChR receptors by these agonists not only facilitates longer-lasting analgesia but also does so without compromising motor coordination. These characteristic positions these compounds as promising candidates for use as analgesics.

Electrophysiology studies were also carried out using the following compounds differing in chirality.

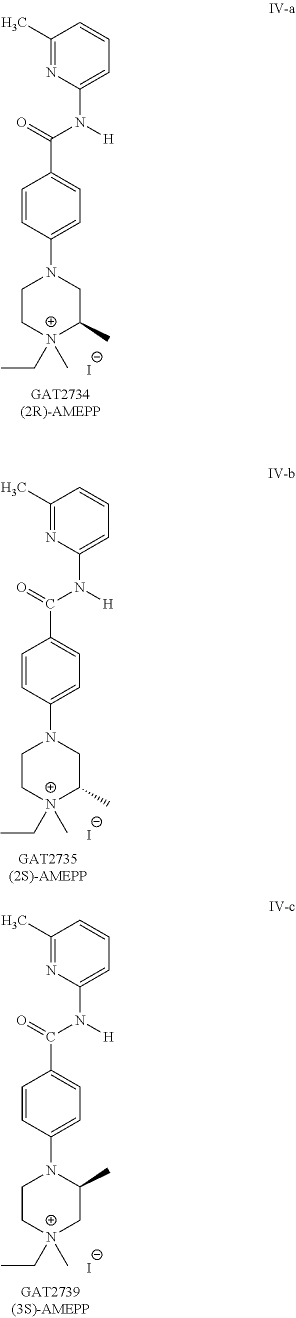

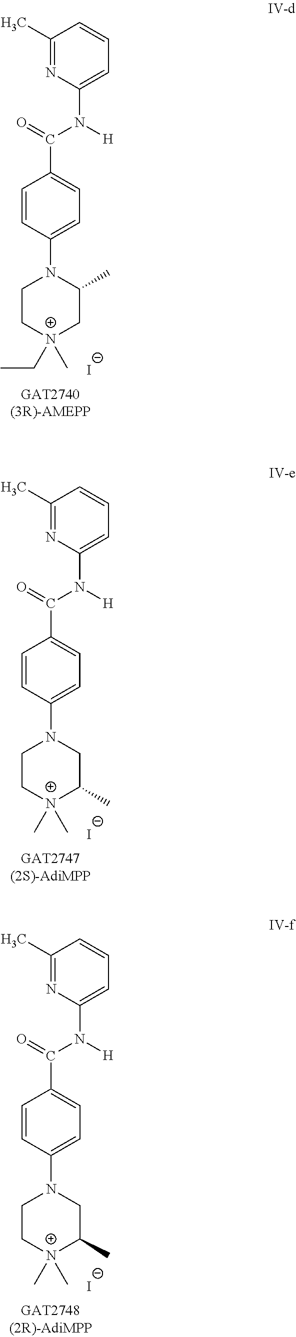

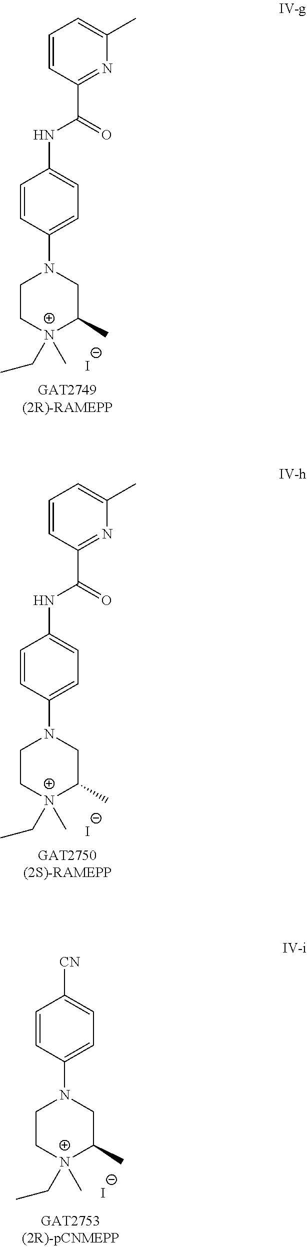

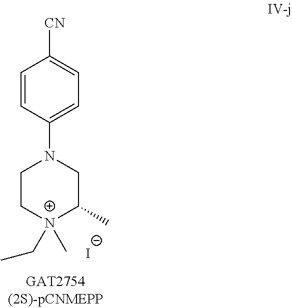 [SMILES: CC[N+]1(C)CCN(c2ccc(C#N)cc2)C[C@@H]1C ; [I-]]

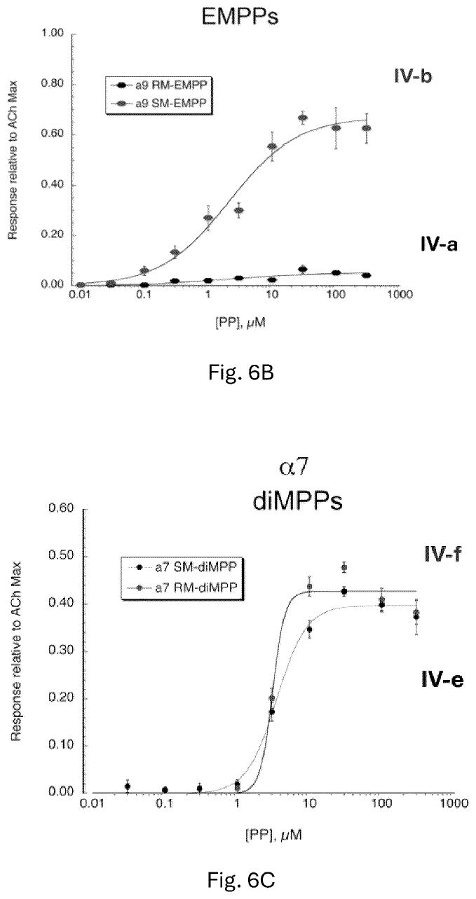

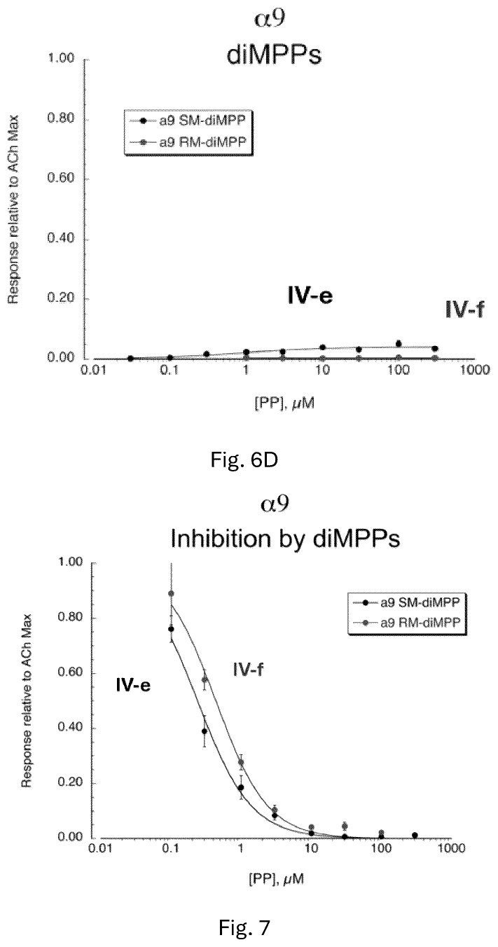

Xenopus oocytes were injected with RNAs to produce functional nAChR which were characterized by two-electrode voltage-clamp measurements. FIGS. 6A-6D and 7 present concentration-response curves (CRCs) for the new compounds tested with human α9, and α9 α10 vs α7 receptors. Table 2 presents the values for Iₘₐₓ and EC₅₀ with these receptors for new chiral 1,1-dialkyl-4-substituted phenylpiperazinium iodides with α9-containing and α7 nAChR.

The SAR data (Table 2) shows that introduction of chirality to the piperazine pharmacophore was helpful in switching the selectivity between α9 vs α7. One enantiomer, i.e., 2S-methyl on piperazine ring (Compound IVb, GAT2735) was selective towards α9, while its 2R enantiomer (Compound Iva, GAT2734) was selective towards α7 (FIGS. 6A, 6B, Table 2). However, when both enantiomers are positioned adjacent to a dimethyl quaternary nitrogen, (Compound IV-e. SM-DIMPP), IV-f (RM-DIMPP), the compounds transition from agonists into antagonists of the alpha 9 nicotinic acetylcholine receptor (nAChR).” (FIGS. 6C, 6D).

TABLE 2
Iₘₐₓ and EC₅₀ values of chiral compounds on α9-containing and α7 nAChR.

| Compd. | | α9 | | α7 | |
|---|---|---|---|---|---|
| No. | Structures | Iₘₐₓ^{a} | EC₅₀^{b} | Iₘₐₓ | EC₅₀ |
| IV-a GAT2734 RM-EMPP | 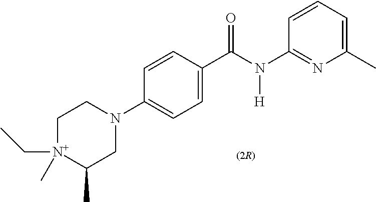 [SMILES: CC[N+]1(C)CCN(c2ccc(C(=O)Nc3cccc(C)n3)cc2)C[C@H]1C] | 0.0521 ± 0.013 | 1.93 ± 2.25 | 0.548 ± 0.02 | 7.16 ± 1.29 |
| IV-b GAT2735 SM-EMPP | 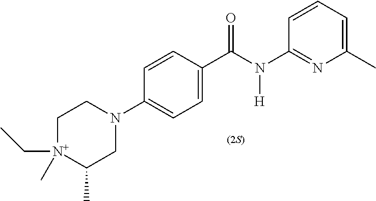 [SMILES: CC[N+]1(C)CCN(c2ccc(C(=O)Nc3cccc(C)n3)cc2)C[C@@H]1C] | 0.670 ± 0.02 | 2.09 ± 0.63 | 0.238 ± 0.004 | 7.10 ± 0.49 |
| IV-e GAT 2747 SM-DIMPP | 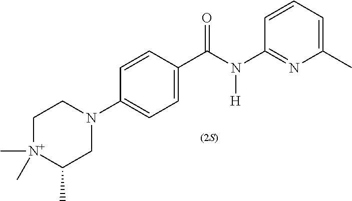 | 0.04 ± 0.005 | 0.8 ± 0.6 | 0.397 ± 0.011 | 3.489 ± 0.35 |
| IV-f GAT 2748 RM-DIMPP | 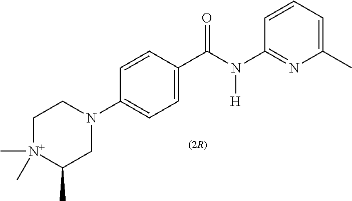 | 0.0065 ± 0.72 | 146.49 ± 60000 | 0.426 ± 0.021 | 3.065 ± 0.38 |
| ACh | | 1.00 ± 0.04 | 26 ± 4 | 1.00 ± 0.04 | 21 ± 3 |

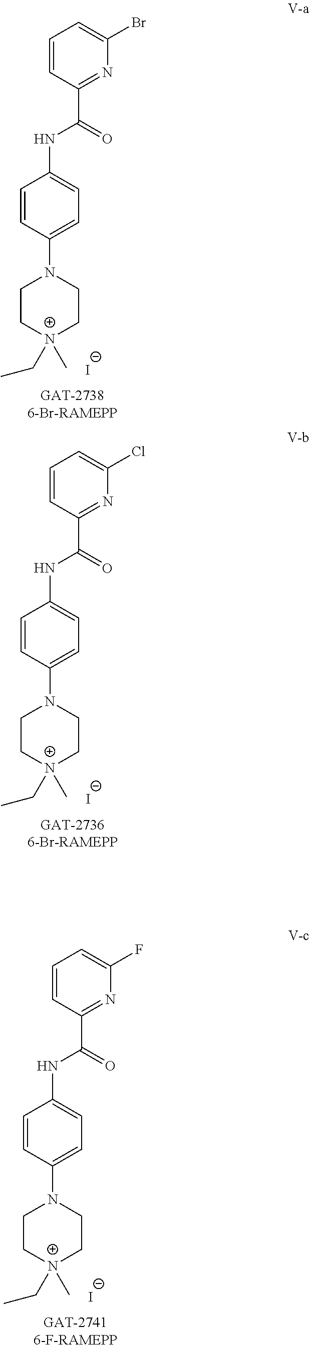 [SMILES: CC[N+]1(C)CCN(c2ccc(NC(=O)c3cccc(Br)n3)cc2)CC1 ; [I-] ; CC[N+]1(C)CCN(c2ccc(NC(=O)c3cccc(Cl)n3)cc2)CC1 ; [I-] ; CC[N+]1(C)CCN(c2ccc(NC(=O)c3cccc(F)n3)cc2)CC1 ; [I-]]

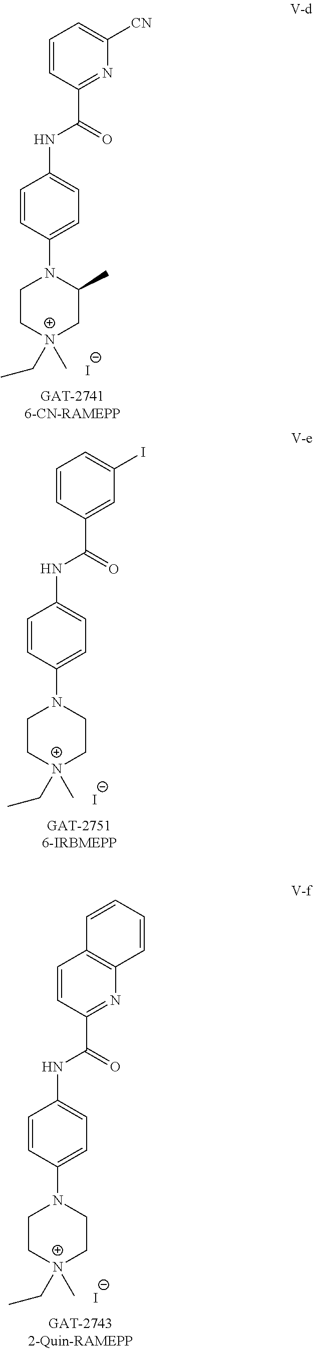 [SMILES: CC[N+]1(C)CCN(c2ccc(NC(=O)c3cccc(C#N)n3)cc2)[C@@H](C)C1 ; [I-] ; CC[N+]1(C)CCN(c2ccc(NC(=O)c3cccc(I)c3)cc2)CC1 ; [I-] ; CC[N+]1(C)CCN(c2ccc(NC(=O)c3ccc4ccccc4n3)cc2)CC1 ; [I-]]

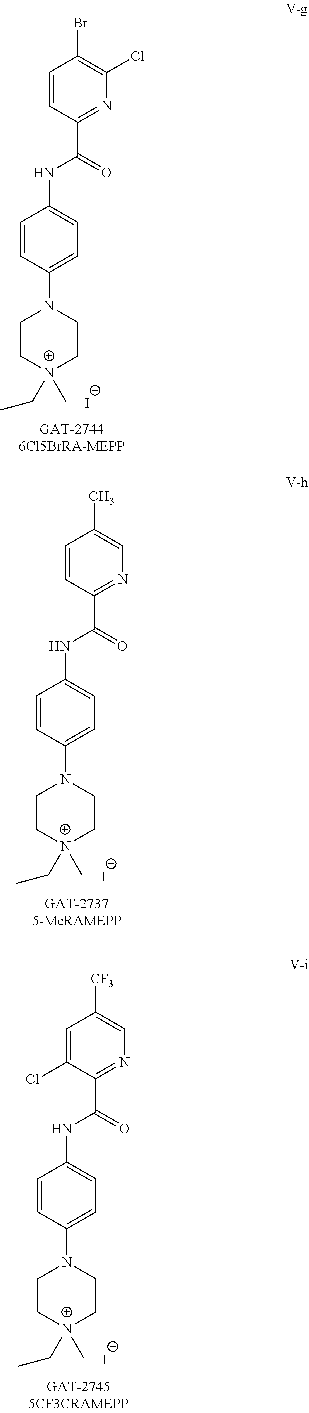 [SMILES: CC[N+]1(C)CCN(c2ccc(NC(=O)c3ccc(Br)c(Cl)n3)cc2)CC1 ; [I-] ; CC[N+]1(C)CCN(c2ccc(NC(=O)c3ccc(C)cn3)cc2)CC1 ; [I-] ; CC[N+]1(C)CCN(c2ccc(NC(=O)c3ncc(C(F)(F)F)cc3Cl)cc2)CC1 ; [I-]]

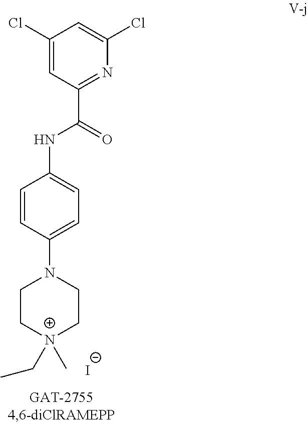 [SMILES: CC[N+]1(C)CCN(c2ccc(NC(=O)c3cc(Cl)cc(Cl)n3)cc2)CC1 ; [I-]]

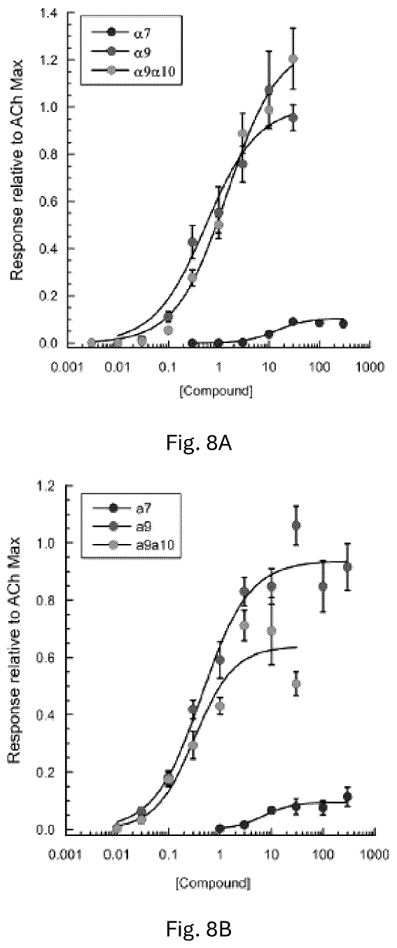

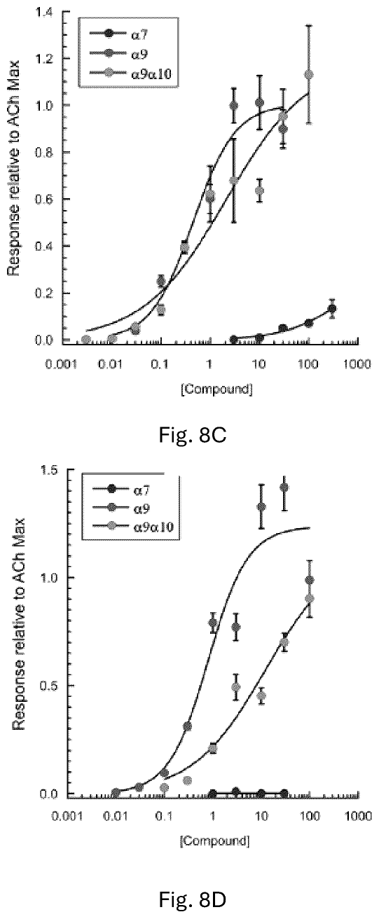

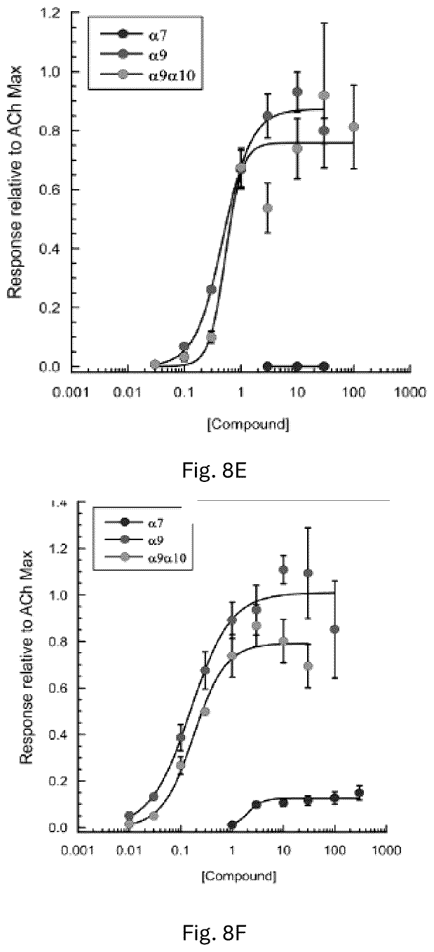

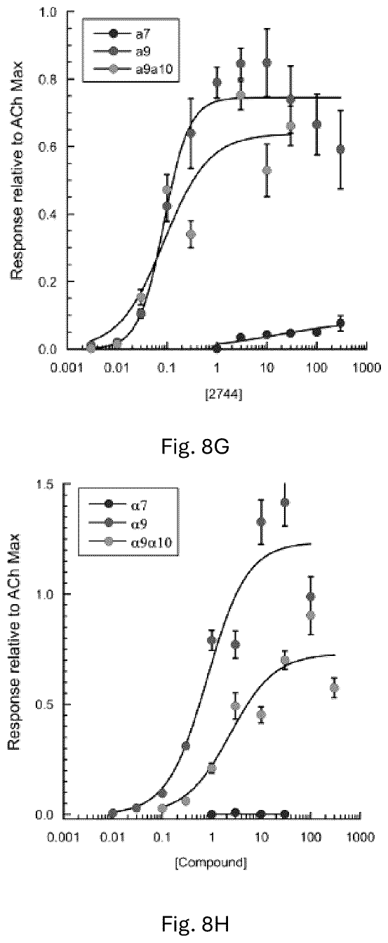

Xenopus oocytes were injected with RNAs to produce functional nAChR which were characterized by two-electrode voltage-clamp measurements. FIGS. 8A-8H presents concentration-response curves (CRCs) for the new compounds tested with human α9, and α9 α10 vs α7 receptors. Table 3 presents the values for Iₘₐₓ and EC₅₀ with these receptors for new 1,1-dialkyl-4-substituted phenylpiperazinium iodides with head group variations with α9-containing and α7 nAChR. The study aimed to scrutinize the impact of chiral substitution on the relative potencies of reverse amide phenyl piperazinium salts (Va-j) on α9 homomeric and α9α10 heteromeric vs α7 receptor. It was found that 6-CN and 6-F bearing functional groups are completely selective to alpha 9 over alpha 7 nAChR, They are full agonists at two time lower potency than compound II. The most potent compounds were found to be Vg bearing 6-Bromo-5-chloro and Vf bearing quinoline head groups with 86 nM and 157 nM potency. Almost all the compounds in this series are selective for α9 homomeric and α9α10 heteromeric over α7 receptor.

TABLE 3
Iₘₐₓ and EC₅₀ values of new reverse amide GATs on α9*-containing and α7 nAChR

| | α9 | | α9α10 | | α7 | |
|---|---|---|---|---|---|---|
| Structures | Iₘₐₓ^{a} | EC₅₀^{b} | Iₘₐₓ | EC₅₀ | Iₘₐₓ | EC₅₀ |
| I | 1.01 ± 0.03 | 0.23 ± 0.034 | 1.04 ± 0.07 | 0.99 ± 0.21 | 0.41 ± 0.025 | 78.34 ± 11.32 |
| V-a | 0.936 ± 0.04 | 0.44 ± 0.108 | 0.63 ± 0.069 | 0.32 ± 0.14 | 0.094 ± 0.012 | 7.10 ± 3.17 |
| V-b | 1.00 ± 0.18 | 0.56 ± 0.065 | 1.26 ± 0.08 | 1.48 ± 0.017 | 0.10 ± 0.028 | 14.034 ± 0.00 |
| V-c | 1.2 ± 0.13 | 0.81 ± 0.38 | 1.13 ± 0.4 | 11.9 ± 18.3 | 0.000 ± 0.000 | — |
| V-d | 0.87 ± 0.03 | 0.48 ± 0.06 | 0.75 ± 0.06 | 0.54 ± 0.18 | — | — |
| V-f | 1.008 ± 0.048 | 0.157 ± 0.039 | 0.79 ± 0.038 | 0.18 ± 0.036 | 0.124 ± 0.009 | 2.03 ± 0.54 |
| V-g | 0.74 ± 0.03 | 0.086 ± 0.02 | 0.637 ± 0.08 | 0.96 ± 0.5 | 0.083 ± 0.048 | 16.34 ± 43.25 |
| V-h | 1.00 ± 0.08 | 0.44 ± 0.14 | 1.19 ± 0.23 | 2.09 ± 2.21 | 0.266 ± 0.33 | 315.49 ± 958.8 |
| V-i | 1.23 ± 0.13 | 0.81 ± 0.38 | 0.73 ± 0.10 | 2.36 ± 1.53 | 0.001 ± 0.14 | — |
| V-e | YTD | YTD | YTD | YTD | YTD | YTD |
| V-j | YTD | YTD | YTD | YTD | YTD | YTD |
| ACh | 1.00 ± 0.04 | 26 ± 4 | 1.00 ± 0.04 | 30 ± 10 | 0.97 ± 0.04 | 21 ± 3 |

*YTD. Yet to be Determined

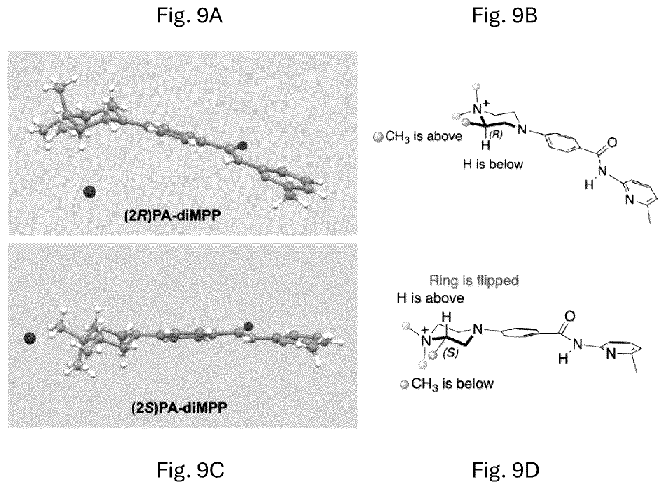

Stereochemistry R and S at the ring position of the piperazine ring of enantiomers of the dimethyl analogs (2R)PA-diMPP and (2S)PA-diMPP was confirmed by obtaining their X-ray crystal structures, which are shown in FIGS. 9A and 9C. The 2R-methyl group occupies the equatorial position, suggesting that the 2S-methyl group should be in the axial position. However, when the crystal structures were analyzed, it was found that, to minimize torsional strain and repulsion, the chair C—C bonds preferred to flip to the opposite chair conformation for greater stability, keeping the 2S methyl at the equatorial position.

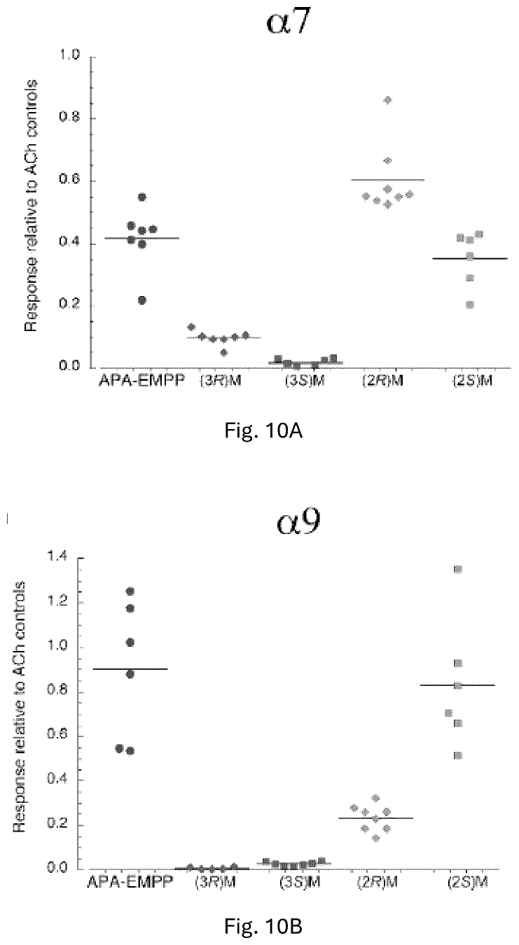

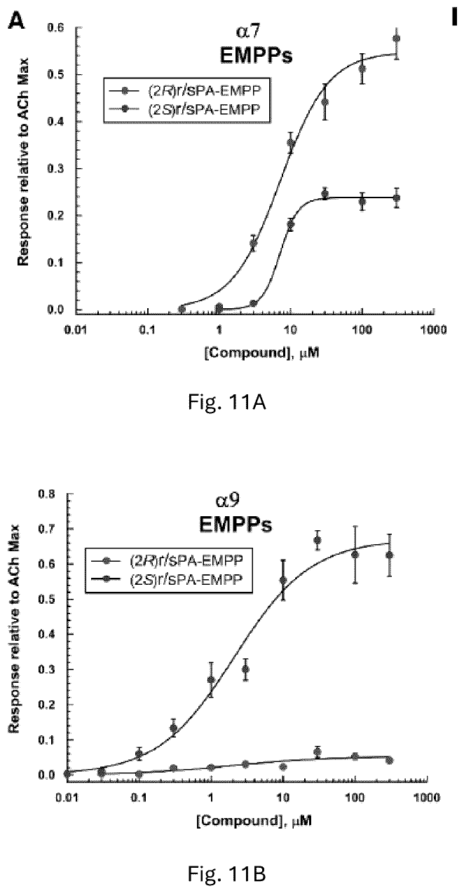

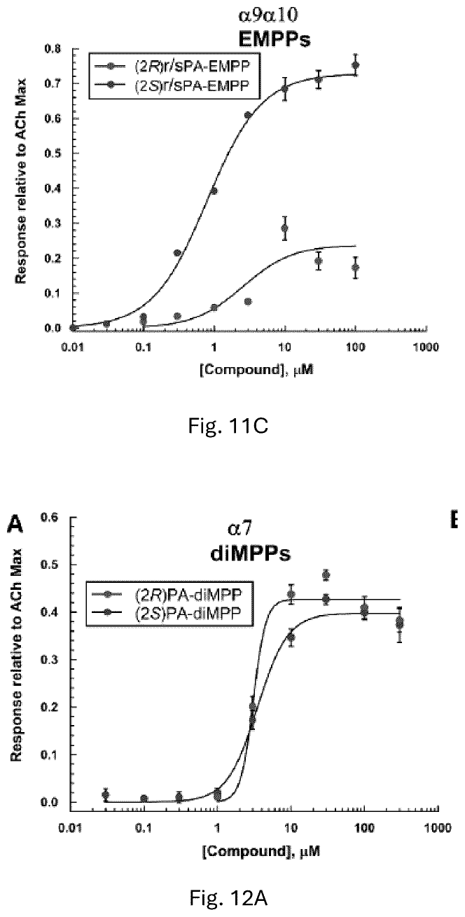

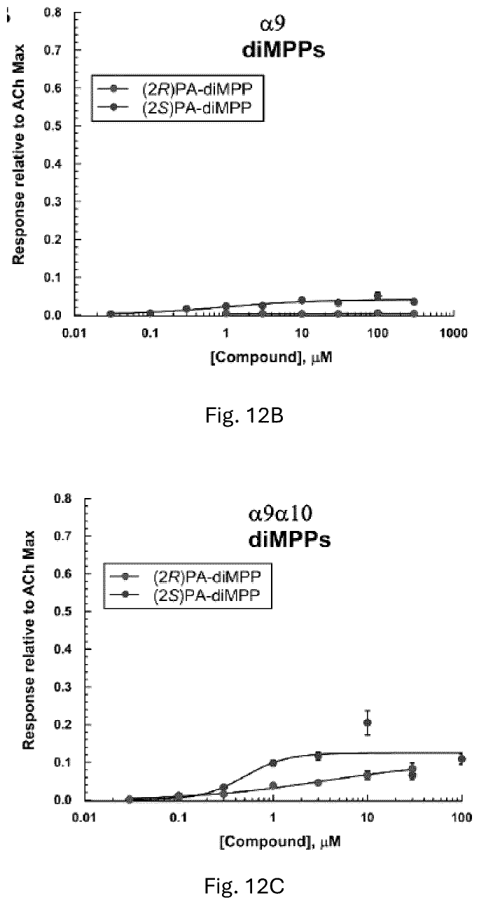

Electrophysiology studies were performed for these enantiomeric compounds. After acquiring initial ACh control responses, each of the isomers and the parent compound PA-EMPP were applied to cells expressing either α7 or α9*. Compared to the parent compound, there was a profound loss of activity for both α7 and α9* receptors with methyl added at the 3 position (Figd. 10A-10B), while activity was largely retained in at least one of the isomers with the methyl at the 2 position. Retention of activity of α7 was best for the 2R isomer and best for α9 with the 2S isomer. Full concentration-response curves were generated for (2R)r/sPA-EMPP and (2S)r/sPA-EMPP for human α9 and α9α10 vs α7 receptor expressing oocytes (FIGS. 11A-11C). The activity of these 2M isomers was consistent with the study shown in FIGS. 10A-10B, with the responses of α9α10 heteromeric receptors similar to those of the homomeric α9 receptors. In order to separate the effects of chirality on the methyls from the potential effects of the chiral nitrogen, chirality was introduced in ring carbons with a dimethyl analog of the parent compound, yielding (2R)PA-diMPP and (2S)PA-diMPP. These compounds lost nearly all agonist activity for α9* receptors, making them selective for α7 (FIGS. 12A-12C). The electrophysiology results are summarized in Table 4.

TABLE 4
Imax and EC50 values for reference and new chiral EMPPs and diMPPs with α9-containing and α7 nAChR.

| | α9 | | α9α10 | | α7 | |
|---|---|---|---|---|---|---|
| Compound | I [symbol] | EC₅₀ [symbol] | I [symbol] | EC₅₀ | I [symbol] | EC₅₀ |
| PA-EMPP | 1.19 ± 0.03 | 0.51 ± 0.07 | 0.92 ± 0.04 | 0.48 ± 0.09 | 0.40 ± 0.02 | 6.1 ± 1.1 |
| (2R)r/sPA-EMPP | 0.052 ± 0.013 | 1.93 ± 2.25 | 0.24 ± 0.13 | 2.54 ± 0.003 | 0.55 ± 0.02 | 7.16 ± 1 [symbol] 9 |
| (2S)r/sPA-EMPP | 0.670 ± 0.02 | 2.09 ± 0.63 | 0.73 ± 0.05 | 0.79 ± 0.009 | 0.24 ± 0.004 | 7.10 ± 0.49 |
| (2R)PA-diMPP | 0.0065 ± 0.72 | N.A. [symbol] | 0.10 ± 0.017 | 3.5 ± 2.35 | 0.426 ± 0.02 | 3.065 ± 0.38 |
| (2S)PA-diMPP | 0.04 ± 0.005 | 0.8 ± 0.6 | 0.12 ± 0.002 | 0.49 ± 0.39 | 0.397 ± 0.01 | 3.489 ± 0.35 |
| ACh | 1.00 ± 0.04 | 26 ± 4 | 1.00 ± 0.04 | 30 ± 10 | 0.97 ± 0.04 | 21 ± 3 |

[symbol]

indicates data missing or illegible when filed

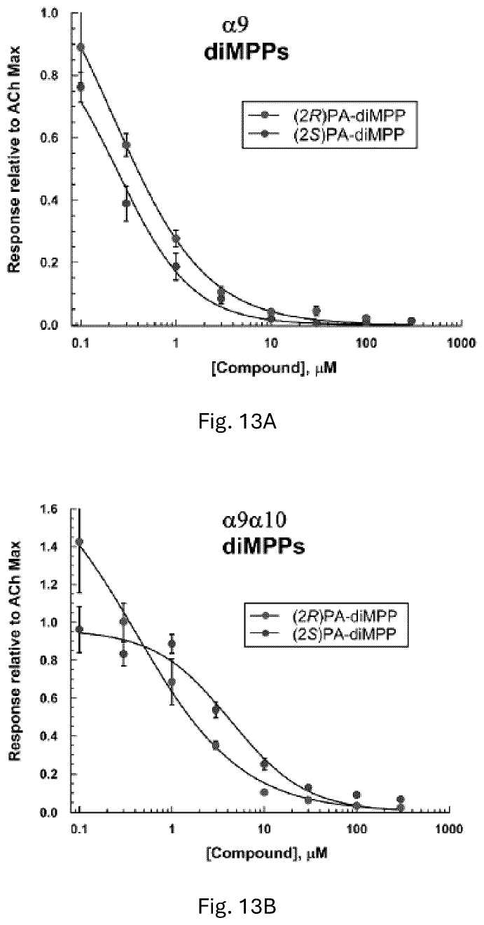

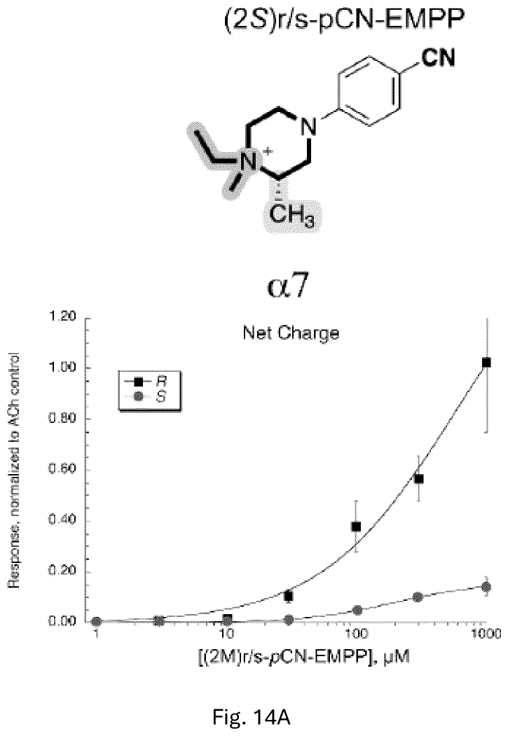

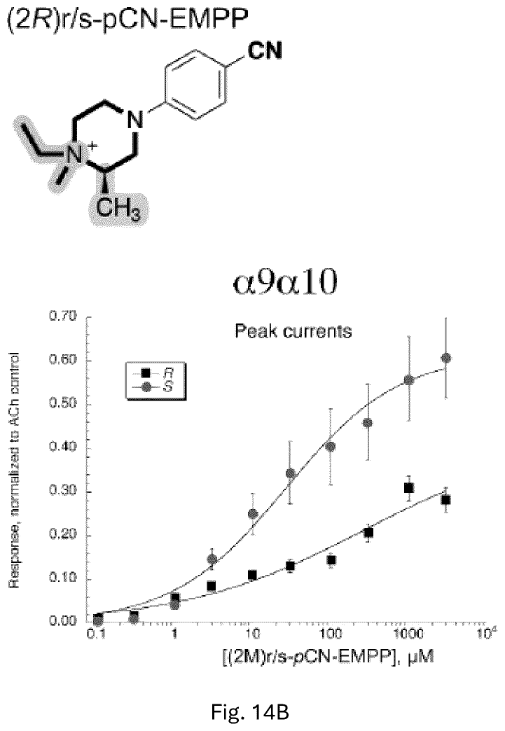

With such low partial agonist activity for α9* receptors, the degree to which they would antagonize ACh-evoked responses was tested in co-application experiments (FIGS. 13A-13B). Experimental responses were measured relative to the preceding ACh control response. The data indicate that both (2R)PA-diMPP and (2S)PA-diMPP functioned as relatively potent antagonists on α9, with IC50 values of 0.21±0.06 μM and 0.24±0.11 μM, and on α9α10 with IC50 values of 0.406±0.15 and 4.33±1.14, respectively. The transition from agonist to antagonist behavior in α7 to α9* nAChRs indicates a slightly bigger dialkyl tail on nitrogen, or perhaps more importantly, the asymmetry of the groups and potential for multiple rotamers is important for α9 agonism, while chirality is more important for α7 selectivity.

Further electrophysiology studies were carried out using chiralized PA-EMPP compounds in which the large arylamido was reduced to a small p-CN group. The results are shown in FIGS. 14A-14B and Table 5.

TABLE 5
EC50 values in units of μM and Imax limits for chiral
pCN-EMPPs with α9α10 and α7 nAChR.

| Compound | Iₘₐₓ | EC₅₀ | Iₘₐₓ | EC₅₀ |
|---|---|---|---|---|
| (2R)r/s-pCN-EMPP | ≤0.05 | 201 ± 12 | ≤0.30 | N.A.^{c} |
| (2S)r/s-pCN-EMPP | ≤0.31 | N.A.^{c} | ≤0.61 | 26.6 ± 9.6 |

The results with the chiral methyls were qualitatively similar to those obtained with the PA-EMPP compounds. The R isomer was more effective for α7, and the S isomer was more effective for α9α10. However, as with the PA-EMPP compounds, the potential importance of the chiral nitrogen could not be ascertained.

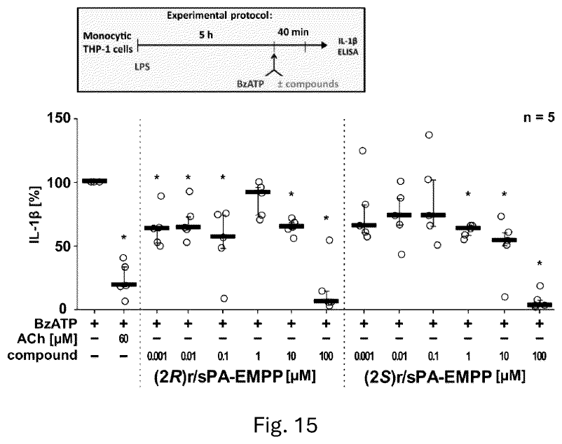

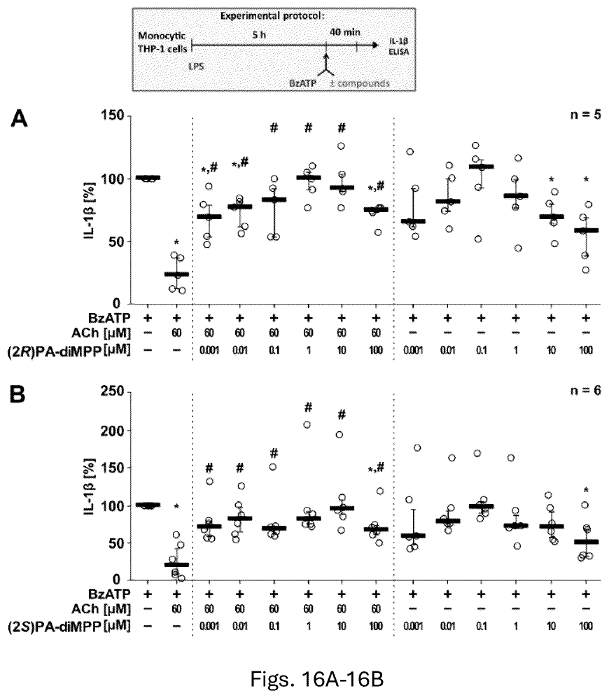

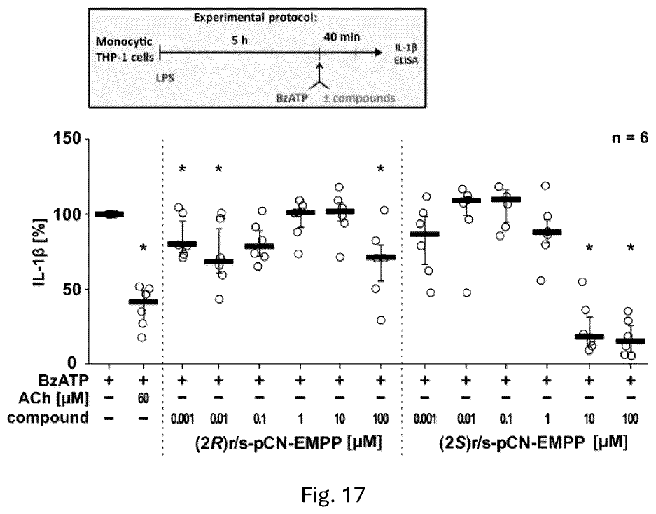

Monocytic THP-1 cells were primed with lipopolysaccharide (LPS, 1 μg/ml) for 5 hours and further stimulated them with BzATP (100 μg/ml) for 40 minutes to induce the maturation and release of IL-1β. The primed THP-1 cells released IL-1β in response to BzATP (in the range of 12 μg/ml to 177 μg/ml, see FIGS. 15, 16A-16B, and 17). When ACh (10 μM) was applied shortly before BzATP as a positive control, the IL-1β concentrations in cell culture supernatants were significantly reduced (FIGS. 9, 10, 11). All compounds tested, (2R)r/sPA-EMPP (FIG. 15), (2S)r/sPA-EMPP (FIG. 15), (2R)r/sPA-diMPP (FIG. 16A), (2S)r/sPA-diMPP (FIG. 16B), (2R)r/s-pCN-EMPP (FIG. 17), and (2S)r/s-pCN-EMPP (FIG. 17), significantly inhibited the BzATP-induced release of IL-1β. For all compounds, the 100 μM concentration seemed to be most effective. Only (2R)r/sPA-EMPP (FIG. 15) and (2R)r/s-pCN-EMPP (FIG. 16A) provoked moderate but significant inhibitory effects already at the 1 nM concentration, which was the lowest concentration tested. Hence, these compounds seemed to function as agonists at monocytic nAChRs. In addition, (2R)r/sPA-diMPP (FIG. 16A) and (2S)r/sPA-diMPP (FIG. 16B) significantly antagonized the effect of ACh at all concentrations investigated (1 nM to 100 μM).

The compounds of the present technology can be used to treat, prevent or alleviate to any degree, or diagnose any disease or medical condition (collectively “disorders”) associated with, caused by, or resulting from the action or lack of action of a nAChR containing one or more subunits selected from the group consisting of α9, α10, α7, and combinations thereof. Preventing or alleviating to any degree by a compound of the present technology means reducing the likelihood of occurrence or a severity of any symptom of the disease or medical condition, compared to not administering the compound, by any amount from about 10% to 100%.

The following disorders are examples of disorders that can be treated, prevented, alleviated, or diagnosed using compounds according to the present technology: sensory and auditory disorders; hearing loss (including noise-induced, age-related, or ototoxic); tinnitus; pain; inflammation; neuropathic pain; chronic pain (including inflammatory, musculoskeletal, cancer-induced); visceral pain (including interstitial cystitis and irritable bowel syndrome); neurodegenerative disorders; neurological disorders; multiple sclerosis; Parkinson's disease; peripheral neuropathy; autoimmune disorders; rheumatoid arthritis; Inflammatory bowel disease (including Crohn's disease, ulcerative colitis); cancer (including cancer chemotherapy and pain related to oral, bone, or visceral cancers; chemotherapy-induced hearing loss or neuropathy; preventive care related to platinum-based or taxane-based chemotherapies; and cancer immunomodulation.

### Examples

### Example 1. Synthesis of Reverse Amide Compounds I to III

Chemistry. The general approach used for the synthesis of N,N-dialkyl-4-(substituted phenylpiperazinium iodides (I-III) is depicted in Schemes 1 and 2.

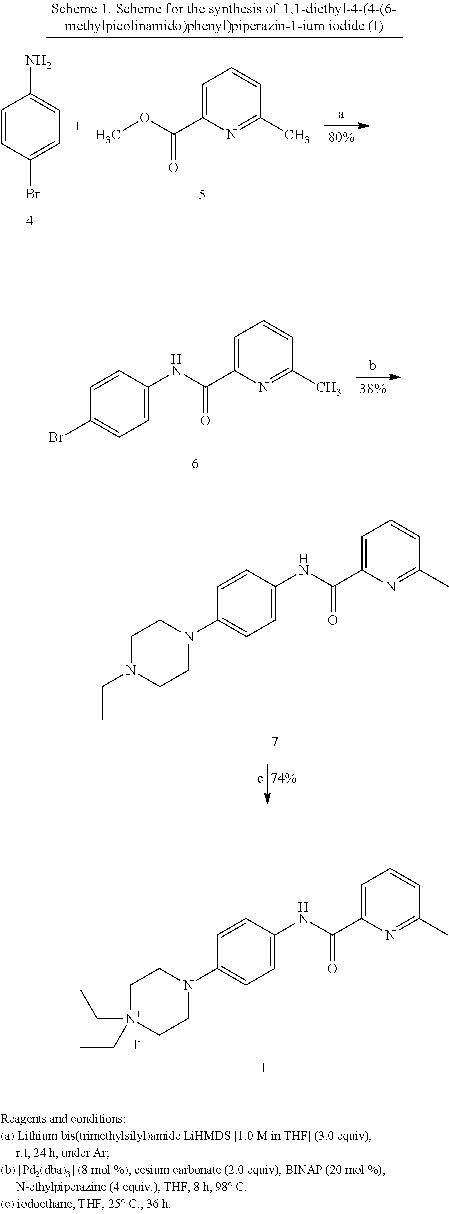

In Scheme 1, for making reverse amide analog I, 4-bromoaniline (4) was made to react with methyl 6-methylpicolinate (5) in the presence of lithium hexamethyldisilazide (LiHMDS) to yield N-(4-bromophenyl)-6-methylpicolinamide (6). The reaction proceeded in 80% yield at room temperature. This transition-metal-free amidation was carried out using arylamines (2.0 equiv), lithium hexamethyldisilazide (LiHMDS) 3.0 equiv, as a base and tetrahydrofuran as a solvent at ambient conditions in the presence of argon. The reactions proceeded rapidly and in high yields. N-(4-bromophenyl)-6-methylpicolinamide (6) was further reacted under Buchwald-Hartwig amination to yield N-(4-(4-ethylpiperazin-1-yl)phenyl)-6-methylpicolinamide (7). However, the reverse amide analog in this case ended up with lower yield (38%). The coupled compound was further treated with iodoethane to yield 1,1-diethyl-4-(4-(6-methylpicolinamido)phenyl)piperazin-1-ium iodide (I).

### Synthesis of N-(4-(4-Ethylpiperazin-1-yl)phenyl)-6-methylpicolinamide (Compound 7)

In a sealed vial purged with nitrogen, N-(4-bromophenyl)-6-methylpicolinamide (6) (0.7 g, 2.4 mmol), Pd₂(dba)₃ (0.18 g, 0.19 mmol (8 mol %)), (2,2′-bis(diphenylphosphino)-1,1′-binaphthyl ligand (BINAP) (0.29 g, 0.48 mmol, 20 mol %), 1-ethylpiperazine (1.2 mL, 9.6 mmol), and Cs₂CO₃ (1.56 g, 4.8 mmol) were dissolved in 3 mL THF and stirred at 98° C. for 10 h. The reaction mixture was evaporated, and the residue was dissolved in dichloromethane, filtered over celite, concentrated, and purified by column chromatography on silica gel, eluting with 90% ethyl acetate in hexane. The fractions containing the desired product were combined, evaporated, and dried under high vacuum. Compound 7 was obtained as a pale yellow solid (0.3 g, 0.92 mmol, yield: 38%). TLC Rf=0.22 (5% methanol in ethyl acetate). ¹H NMR (500 MHz, Chloroform-d) δ 9.94 (s, 1H), 8.08 (d, J=7.6 Hz, 1H), 7.75 (t, J=7.7 Hz, 1H), 7.71-7.65 (m, 2H), 7.29 (d, J=7.7 Hz, 1H), 6.99-6.93 (m, 2H), 3.29-3.11 (m, 4H), 2.62 (s, 3H), 2.58 (t, J=5.0 Hz, 4H), 2.35 (s, 3H). ¹³C NMR (126 MHz, Chloroform-d) δ 161.83, 157.03, 149.33, 148.13, 137.68, 130.41, 125.93, 120.85, 119.31, 116.60, 55.11, 49.49, 46.14, 24.27, 7.21.

### Synthesis of 1,1-Diethyl-4-(4-(6-methylpicolinamido)phenyl)piperazin-1-ium iodide (Compound I)

In a sealed vial, the coupled compound 7 (0.2 g, 0.61 mmol, 1 eq) was dissolved in 0.5 mL dry THF and iodoethane (0.50 mL, 6.16 mmol, 10 eq.) was added; the resulting mixture was stirred at 25° C. for 48 hours until complete consumption of the starting material (TLC in ethylacetate:methanol (7:3), LC-MS. Upon completion of the reaction, hexane was added to the reaction mixture to remove excess iodoethane. The hexane solution was pipetted out from the mixture leaving behind a residue that was dissolved in DCM, rotavaped and recrystallized with ethylacetate. Pure solid was dried under high vacuum to afford I (0.22 g, 0.45 mmol, 74%) as a light-yellow solid. TLC Rf=0.11 in ethyl acetate:methanol (7:3). ¹H NMR (500 MHz, Methanol-d₄) δ 7.99 (d, J=7.7 Hz, 1H), 7.88 (td, J=1.4, 7.7 Hz, 1H), 7.75-7.69 (m, 2H), 7.47 (d, J=7.7 Hz, 1H), 7.12-7.05 (m, 2H), 3.64 (t, J=5.1 Hz, 4H), 3.55 (q, J=7.0 Hz, 8H), 2.65 (s, 3H), 1.37 (t, J=7.3 Hz, 6H). ¹³C NMR (126 MHz, Methanol-d₄). δ 164.46, 159.21, 150.42, 147.91, 139.22, 132.75, 127.61, 122.79, 120.39, 118.03, 58.72, 54.17, 44.22, 24.21, 7.30.

Owing to the lower yield of piperazinyl coupled product in step b in Scheme 1, the synthetic route was changed for compounds II and III (Scheme 2). The synthesis started with 4-nitroiodobenzene. First 4-nitroiodobenzene (8) was coupled with methyl piperazine using Buchwald-Hartwig amination yield to 1-methyl-4-(4-nitrophenyl)piperazine (9) in 82% yield, followed by the reduction of nitro functionality using Raney Ni and hydrogen gas in methanol/tetrahyrofuran leading to the formation of 4-(4-methylpiperazin-1-yl) aniline (10) in 91% yield. 10 was then utilized for the aminolysis of esters 5, 11 using LiHMDS in THF yielding amides 12 and 13 which were then alkylated using iodoethane to yield the desired piperazinium revere amide salts II and III.

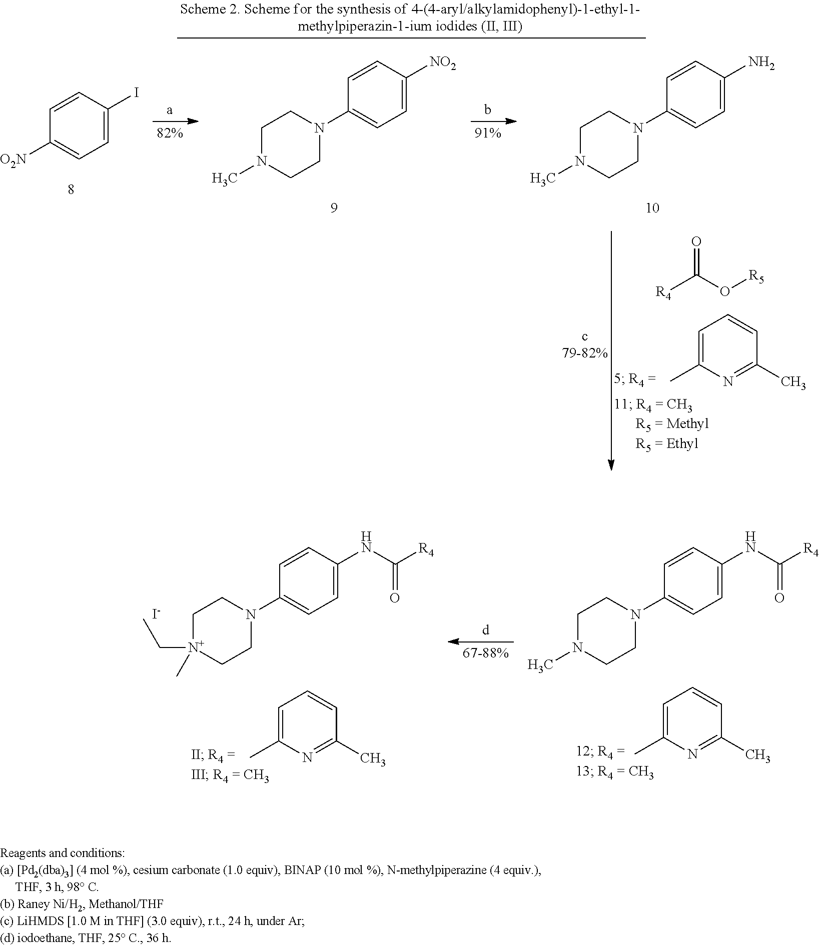

### Synthesis of 1-Methyl-4-(4-nitrophenyl)piperazine (Compound 9)

In a sealed vial purged with nitrogen, 4-nitroiodobenzene (1.5 g, 6.02 mmol), Pd₂(dba)₃ (0.22 g, 0.24 mmol (4 mol %)), (2,2′-bis(diphenylphosphino)-1,1′-binaphthyl ligand (BINAP) (0.37 g, 0.60 mmol, 10 mol %), 1-methylpiperazine (2 mL, 18.7 mmol), and Cs₂CO₃ (1.96 g, 6.02 mmol) were dissolved in 3 mL THF and stirred at 98° C. for 3 h. The reaction mixture was evaporated, and the residue was dissolved in dichloromethane, filtered over celite, concentrated, and purified by column chromatography on silica gel, eluting with ethyl acetate. The fractions containing the desired product were combined, evaporated, and dried under high vacuum. Compound 9 was obtained as an orange solid (1.1 g, 2.3 mmol, yield: 82.5%). TLC Rf=0.76 in 5% methanol in ethyl acetate. ¹H NMR (500 MHz, Chloroform-d) δ 8.11 (d, J=9.3 Hz, 2H), 6.82 (d, J=9.4 Hz, 2H), 3.49-3.40 (m, 4H), 2.58-2.48 (m, 4H), 2.35 (s, 3H). ¹³C NMR (126 MHz, Chloroform-d) δ 154.83, 138.43, 132.12, 128.54, 125.93, 112.66, 54.53, 46.99, 46.07.

### Synthesis of 4-(4-Methylpiperazin-1-yl) aniline (Compound 10)

Raney Nickel (0.02 g, 8 mol %) was added in a mixture of 1-methyl-4-(4-nitrophenyl)piperazine (9) (1 g, 4.52 mmol) of in methanol/THF 8 mL (1:1) in a three neck round bottom flask equipped with a supply of hydrogen gas contained in a balloon. The reaction is allowed to stir, and hydrogen gas is released at a slow rate. The reaction mixture is allowed to stir for 2 hours at room temperature. After the completion of reaction, the reaction mixture is diluted with dichloromethane passed through celite and concentrated, recrystallized with DCM: Hexane and dried under high vacuum. Compound 10 was obtained as a brown solid (0.79 g, 4.13 mmol, yield: 91.3% yield). TLC Rf=0.25 in 2% methanol in ethyl acetate. ¹H NMR (500 MHz, Chloroform-d) δ 6.83-6.77 (m, 2H), 6.66-6.60 (m, 2H), 3.33 (d, J=74.1 Hz, 2H), 3.11-3.00 (m, 4H), 2.62-2.50 (m, 4H), 2.33 (s, 3H). ¹³C NMR (126 MHz, Chloroform-d) δ 144.48, 140.11, 118.54, 116.19, 55.34, 50.86, 46.16.

### Synthesis of N-(4-(4-Methylpiperazin-1-yl)phenyl)-6-methylpicolinamide (Compound 12)

An oven-dried round bottom flask was flushed with argon and charged with 4-(4-methylpiperazin-1-yl) aniline (10) (0.75 g, 3.92 mmol, 1.1 equiv). Lithium bis(trimethylsilyl)amide (LiHMDS) 1.0 M in THF, 3.0 equiv (10.5 mL, 10.5 mmol) was added with vigorous stirring at room temperature, followed by dropwise addition of methyl 6-methylpicolinate (5) (neat, 0.51 mL, 3.52 mmol 1.0 equiv) and the reaction mixture was stirred at room temperature. After completion of the reaction (monitored by TLC), the reaction mixture was quenched with aqueous ammonium chloride (1.0 M, 10 mL), then diluted with dichloromethane (30 mL), and the resulting the organic layer was washed with water (1×40 mL), brine (1×40 mL), then dried and concentrated. Crude was purified by reverse phase column chromatography and the product eluted at 100% water containing 0.1% formic acid. The fractions containing product were neutralized with sodium bicarbonate followed by extraction again with DCM. 0.9 g, 2.89 mmol of N-(4-(4-methylpiperazin-1-yl)phenyl)-6-methylpicolinamide (12). Yield: 82.1%; TLC Rf=0.19 in Ethyl acetate:Methanol (9:1). ¹H NMR (500 MHz, Chloroform-d) δ 9.94 (s, 1H), 8.08 (d, J=7.6 Hz, 1H), 7.75 (t, J=7.7 Hz, 1H), 7.71-7.65 (m, 2H), 7.29 (d, J=7.7 Hz, 1H), 6.99-6.93 (m, 2H), 3.29-3.11 (m, 4H), 2.62 (s, 3H), 2.58 (t, J=5.0 Hz, 4H), 2.35 (s, 3H). ¹³C NMR (126 MHz, Chloroform-d) δ 161.83, 157.03, 149.33, 148.13, 137.68, 130.41, 125.93, 120.85, 119.31, 116.60, 55.11, 49.49, 46.14, 24.27.

### Synthesis of N-(4-(4-Methylpiperazin-1-yl)phenyl) acetamide (Compound 13)

An oven-dried round bottom flask was flushed with argon and charged with 4-(4-methylpiperazin-1-yl) aniline (10) (0.70 g, 3.65 mmol, 1.1 equiv). Lithium bis(trimethylsilyl)amide (LiHMDS) 1.0 M in THF, 2.0 equiv (6.57 mL, 6.5 mmol) was added with vigorous stirring at room temperature, followed by dropwise addition of ethylacetate (neat, 0.32 mL, 3.29 mmol 1.0 equiv) and the reaction mixture was stirred at room temperature. After completion of the reaction (monitored by TLC), the reaction mixture was quenched with aqueous ammonium chloride (1.0 M, 10 mL), then diluted with dichloromethane (30 mL), and the resulting the organic layer was washed with water (1×40 mL), brine (1×40 mL), then dried and concentrated. Crude was purified by reverse phase column chromatography and the product eluted at 100% water containing 0.1% formic acid. The fractions containing product were neutralized with sodium bicarbonate followed by extraction again with DCM. 0.61 g, 2.61 mmol of N-(4-(4-methylpiperazin-1-yl)phenyl) acetamide (13). Yield: 79.4%; TLC Rf=0.16 in Ethyl acetate:Methanol (9:1). ¹H NMR (500 MHz, Chloroform-d) δ 7.35 (d, J=8.4 Hz, 2H), 6.86 (d, J=7.9 Hz, 2H), 3.15 (s, 4H), 2.59-2.54 (m, 5H), 2.34 (s, 3H), 2.12 (s, 3H). ¹³C NMR (126 MHz, Chloroform-d) δ 168.20, 148.29, 130.39, 121.52, 116.63, 55.02, 49.44, 46.05, 24.37.

### Synthesis of 1-Ethyl-1-methyl-4-(4-(6-methylpicolinamido)phenyl)piperazin-1-ium iodide (Compound II)

In a sealed vial, the coupled compound 12 (0.25 g, 0.80 mmol, 1 equiv) was dissolved in 0.5 mL dry THF and iodoethane (0.64 mL, 8.05 mmol, 10 equiv) was added; the resulting mixture was stirred at 25° C. for 48 hours until complete consumption of the starting material (TLC in ethylacetate:methanol (7:3). Upon completion of the reaction, hexane was added to the reaction mixture to remove excess iodoethane. The hexane solution was pipetted out from the mixture leaving behind a residue that was dissolved in DCM, rotavaped and recrystallized with ethylacetate. Pure solid was dried under high vacuum to afford II (0.33 g, 0.70 mmol, 87.8%) as a light-yellow solid. TLC Rf=0.2 in ethyl acetate:methanol (7:3). ¹H NMR (500 MHz, Methanol-d₄) δ 7.98 (d, J=7.7 Hz, 1H), 7.87 (t, J=7.7 Hz, 1H), 7.77-7.66 (m, 2H), 7.46 (d, J=7.7 Hz, 1H), 7.09 (d, J=8.7 Hz, 2H), 3.71-3.39 (m, 10H), 3.18 (s, 3H), 2.64 (s, 3H), 1.44 (t, J=7.3 Hz, 3H). ¹³C NMR (126 MHz, Methanol-d₄) δ 164.42, 159.18, 150.42, 147.89, 139.19, 132.79, 127.58, 122.77, 120.37, 118.16, 61.04, 60.83, 46.65, 44.57, 24.21, 7.73.

### Synthesis of 4-(4-Acetamidophenyl)-1-ethyl-1-methylpiperazin-1-ium Iodide (Compound III)

In a sealed vial, N-(4-(4-methylpiperazin-1-yl)phenyl) acetamide (13) (0.25 g, 1.07 mmol, 1 equiv) was dissolved in 0.5 mL dry THF and iodoethane (0.86 mL, 10.7 mmol, 10 equiv) was added; the resulting mixture was stirred at 25° C. for 24 hours until complete consumption of the starting material (TLC in ethylacetate:methanol (7:3) and LC-MS analysis). Upon completion of the reaction, hexane was added to the reaction mixture to remove excess iodoethane. The hexane solution was pipetted out from the mixture leaving behind a residue that was dissolved in DCM, rotavaped and recrystallized with ethylacetate/hexane (9:1). Pure solid was dried under high vacuum to afford compound III (0.28 g, 0.71 mmol, 67.0%) as a light-yellow solid. TLC Rf=0.17 in ethyl acetate:methanol (7:3). ¹H NMR (500 MHz, Methanol-d₄) δ 7.46 (d, J=8.4 Hz, 2H), 7.00 (d, J=8.5 Hz, 2H), 3.59 (qd, J=6.2, 12.2, 12.9 Hz, 10H), 3.16 (s, 3H), 2.09 (s, 3H), 1.42 (d, J=7.1 Hz, 3H). ¹³C NMR (126 MHz, Methanol-d₄) δ 170.02, 146.13, 132.29, 121.16, 116.70, 59.43, 53.42, 48.11, 47.94, 47.77, 47.60, 47.43, 47.26, 47.09, 45.20, 43.27, 22.23, 6.28.

Example 2. Synthesis of N,N-Dialkyl-4-(substituted carbamoyl)phenyl)piperazinium Iodides, Reverse Amide Analogs, and p-CN Analogs.

Chemistry. The general approach used for the synthesis of N,N-dialkyl-4-(substituted carbamoyl)phenyl)piperazinium iodides IVa-f and the reverse amide analogs IVg,h and p-CN analogs IVi,j is depicted in Schemes 3 to 5.

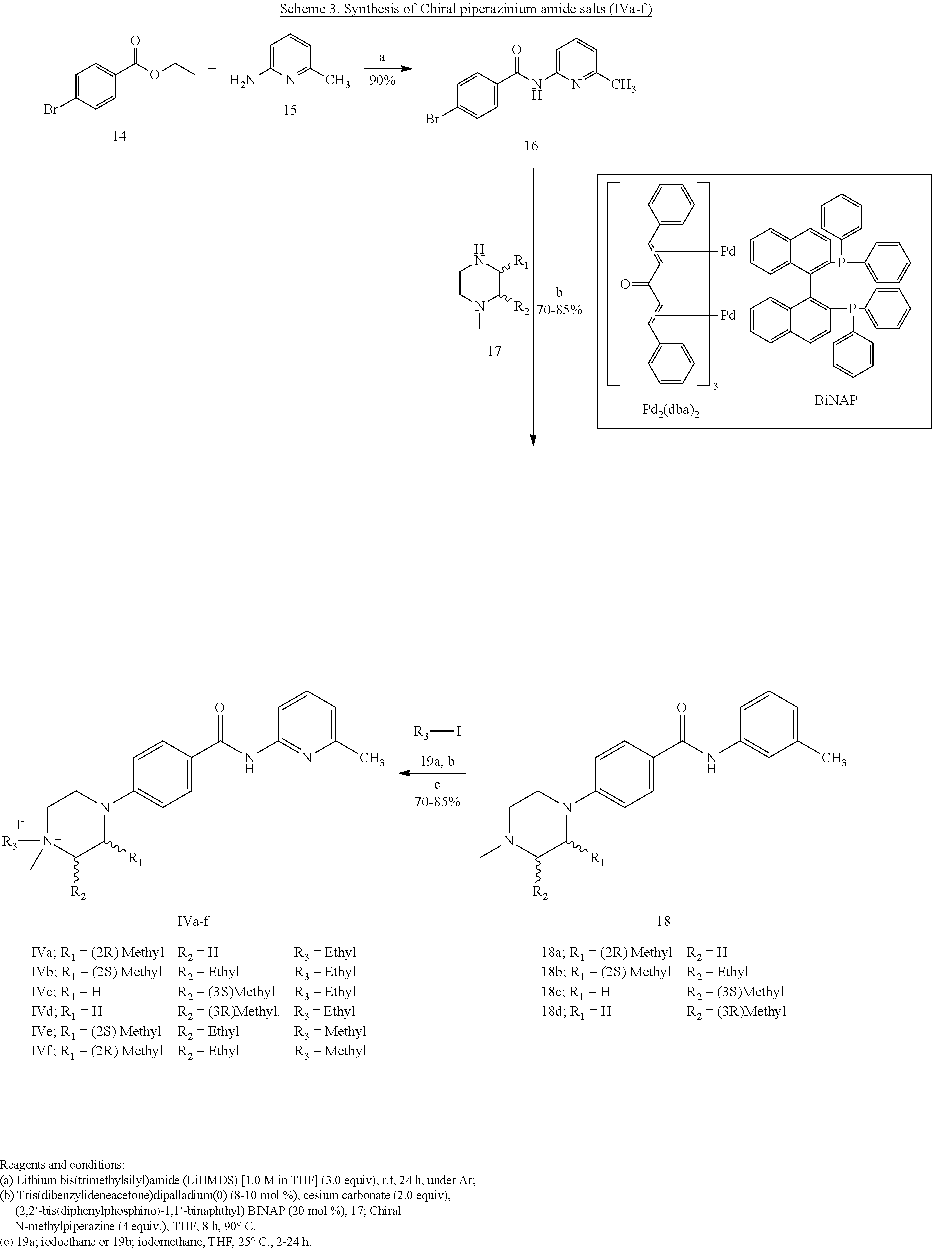

Amidation of p-bromo ethylbenzoate 14 (Scheme 3) formed 4-bromo-picolinamide 16 by acyl substitution with amines (15); these reactions proceeded in excellent 90-96% yield at room temperature. This transition-metal-free amidation was carried out using arylamine (2.0 equiv), lithium hexamethyldisilazide (LiHMDS) 3.0 equiv, as a base and tetrahydrofuran as a solvent at ambient conditions in the presence of argon. The reactions proceeded rapidly and in high yields. The second step was the key step in the synthesis i.e, the C—N cross-coupling reaction for N-arylation of chiral alkylpiperazine R-1,2 or R-1,3-dialkyliperazine (17a,d) or S-1,2 or S-1,3-dialkyliperazine (17b,c) utilizing 4-bromo-picolinamide 16, leading to the formation of N-(2/3,chiral alkyl-N-alkylpiperazin-1-yl)-N-aryl benzamides (18a-d). As previously described ²⁶ Buchwald-Hartwig C—N cross-coupling reactions were superior to Ullman chemistry,²⁸ when the coupling targets included amide functionality. Initially palladium diacetate (10 mol %) and (2,2′-bis(diphenylphosphino)-1,1′-binaphthyl) ligand (BINAP) (20 mol %) in toluene was used. The reaction failed to complete, even after 4 days, leading to exceptionally low yields. Also employed was tetrakis(triphenylphosphine)palladium(0) [palladium tetrakis] (10 mol %) instead of Pd(OAc)₂, using sodium tertiary butoxide (2 equiv) as a base in toluene; however, it ended up with a number of impurities that could not be separated even by column chromatography. Finally, the reaction 8-10 utilizing mol % of tris(dibenzylideneacetone)dipalladium⁰ (Pd₂(dba)₃) in cesium carbonate (2.0 equiv) as a base and BINAP (10-20 mol %) in 1-2 mL of dioxane afforded a good yield (60-78%) of coupled product after purification. Once obtained, the chiral piperazinyl benzamides (18a-d) were then converted into the quaternary ammonium salts by alkylation with ethyl iodide or methyl iodide in tetrahydrofuran and then purified by precipitation to afford the chiral 2 or 3 alkyl-N,N-dialkyl-4-(substituted aryl) carbamoyl)phenyl)piperazinium iodides (IVa-f. The synthesis of chiral 1,1-dialkyl-4-(4-(6-methylpicolinamido)phenyl)piperazin-1-ium iodides (IVg,h) is shown in Scheme 4 and synthesis of chiral 4-(4-cyanophenyl)-1-ethyl-1,2-dimethylpiperazin-1-ium iodide (IV-i,j) is depicted in Scheme 5.

### Synthesis of 4-bromo-N-arylbenzamide (Compound 16)

In a sealed tube, flushed with Ar and charged with 6-methylpyridin-2-amine 15 (1.97 g, 18.2 mmol, 2.0 eq), Lithium bis(trimethylsilyl)amide (LiHMDS) 1.0 M in THF, 3.0 eq (27.3 mL, 27.4 mmol,) was added with vigorous stirring at room temperature, followed by dropwise addition of ethyl 4-bromobenzoate (14) (1.46 mL, 9.12 mmol 1.0 equiv) and the reaction mixture was stirred at room temperature. After completion of the reaction (monitored by TLC), the reaction mixture was quenched with aqueous ammonium chloride (1.0 M, 10 mL), then diluted with dichloromethane (30 mL), and the resulting the organic layer was washed with water (1×30 mL), brine (1×30 mL), then dried and concentrated and dried under vacuum to afford the desired product 16 as a pale white solid (2.43 g, 8.35 mmol, 92% yield), m.p: 73.7-74.3° C., TLC Rf=0.31 in n-hexane:ethyl acetate (6:4). ¹H NMR (600 MHz, Methanol-d₄) δ 8.02 (dd, J=4.6, 8.5 Hz, 1H), 7.88 (ddd, J=1.7, 6.4, 10.3 Hz, 2H), 7.75-7.65 (m, 3H), 7.07-7.01 (m, 1H), 2.49 (s, 3H, CH₃). ¹³C NMR (151 MHz, Methanol-d₄) δ 157.01, 150.89, 138.51, 133.24, 131.55, 129.11, 126.38, 119.33, 111.68, 111.66, 22.59. LCMS (ESI) calculated C₁₃H₁₁BrN₂O [M+H] 291.01, Found. 291.0.

### Synthesis of (R)-4-(3,4-dimethylpiperazin-1-yl)-N-(6-methylpyridin-2-yl)benzamide (Compound 18a)

In a sealed vial purged with nitrogen, bromo compound 16 (0.75 g, 2.58 mmol), Pd₂(dba)₃ (0.18 g, 0.206 mmol (8 mol %)), (2,2′-bis(diphenylphosphino)-1,1′-binaphthyl ligand (BINAP) (0.24 g, 0.386 mmol, 15 mol %), (R)-1,2-dimethylpiperazine (0.88 g, 7.73 mmol), and Cs₂CO₃ (1.26 g, 3.86 mmol) were dissolved in 5 mL THF and stirred at 98° C. for 24 h. The reaction mixture was evaporated, and the residue was dissolved in dichloromethane, filtered over celite, concentrated, and purified by reverse phase column chromatography on silica gel, eluting with water and acetonitrite in formic acid (0.05%). The fractions were collected and combined after neutralization with sodium bicarbonate and extracting with DCM. The fractions containing the desired product were combined, evaporated, and dried under high vacuum. Compound 18a was obtained as a pale liquid that solidify on cooling (0.645 g, 1.99 mmol, yield: 77%). TLC Rf=0.34 in 5% methanol in ethyl acetate.

### Synthesis of(S)-4-(3,4-dimethylpiperazin-1-yl)-N-(6-methylpyridin-2-yl)benzamide (Compound 18b)

The compound 18b was prepared from 16 (0.800 g, 2.75 mmol) and (R)-1,2-dimethylpiperazine (0.88 g, 7.73 mmol) using the same procedure as for compound 18a. Compound 18b was obtained as a pale gummy liquid (0.62 g, 1.91 mmol, yield: 74.19%). TLC Rf=0.34 in 5% methanol in ethyl acetate.

### Synthesis of(S)-4-(2,4-dimethylpiperazin-1-yl)-N-(6-methylpyridin-2-yl)benzamide (Compound 18c)

The compound 18b was prepared from 16 (0.75 g, 2.58 mmol) and (R)-1,2-dimethylpiperazine (0.94 g, 8.24 mmol) using the same procedure as for compound 18a. Compound 18b was obtained as a pale gummy liquid (0.70 g, 2.16 mmol, yield: 78.5%). TLC Rf=0.37 in 5% methanol in ethyl acetate.

### Synthesis of (R)-4-(2,4-dimethylpiperazin-1-yl)-N-(6-methylpyridin-2-yl)benzamide (Compound 18d)

The compound 18d was prepared from 16 (0.75 g, 2.58 mmol) and (R)-1,2-dimethylpiperazine (0.88 g, 7.73 mmol) using the same procedure as for compound 18a. Compound 18b was obtained as a white solid (0.67 g, 2.07 mmol, yield: 80.17%). TLC Rf=0.33 in 5% methanol in ethyl acetate.

### Synthesis of (2R)-1-ethyl-1,2-dimethyl-4-(4-((6-methylpyridin-2-yl)carbamoyl)phenyl)piperazin-1-ium iodide (Compound IV-a)

In a sealed vial, (R)-4-(3,4-dimethylpiperazin-1-yl)-N-(6-methylpyridin-2-yl)benzamide 18a (0.15 g, 0.462 mmol, 1 equiv) was dissolved in 0.5 mL dry THF and iodoethane (0.37 mL, 4.62 mmol, 10 equiv) was added; the resulting mixture was stirred at 25° C. for 24 hours until complete consumption of the starting material (TLC in ethylacetate:methanol (7:3) and LC-MS analysis). Upon completion of the reaction, hexane was added to the reaction mixture to remove excess iodoethane. The hexane solution was pipetted out from the mixture leaving behind a residue that was dissolved in DCM, rotavaped and recrystallized with ethylacetate/hexane (9:1). Pure solid was dried under high vacuum to afford compound III (0.165 g, 0.343 mmol, 74.2.0%) as a white solid. TLC Rf=0.17 in ethyl acetate:methanol (7:3).

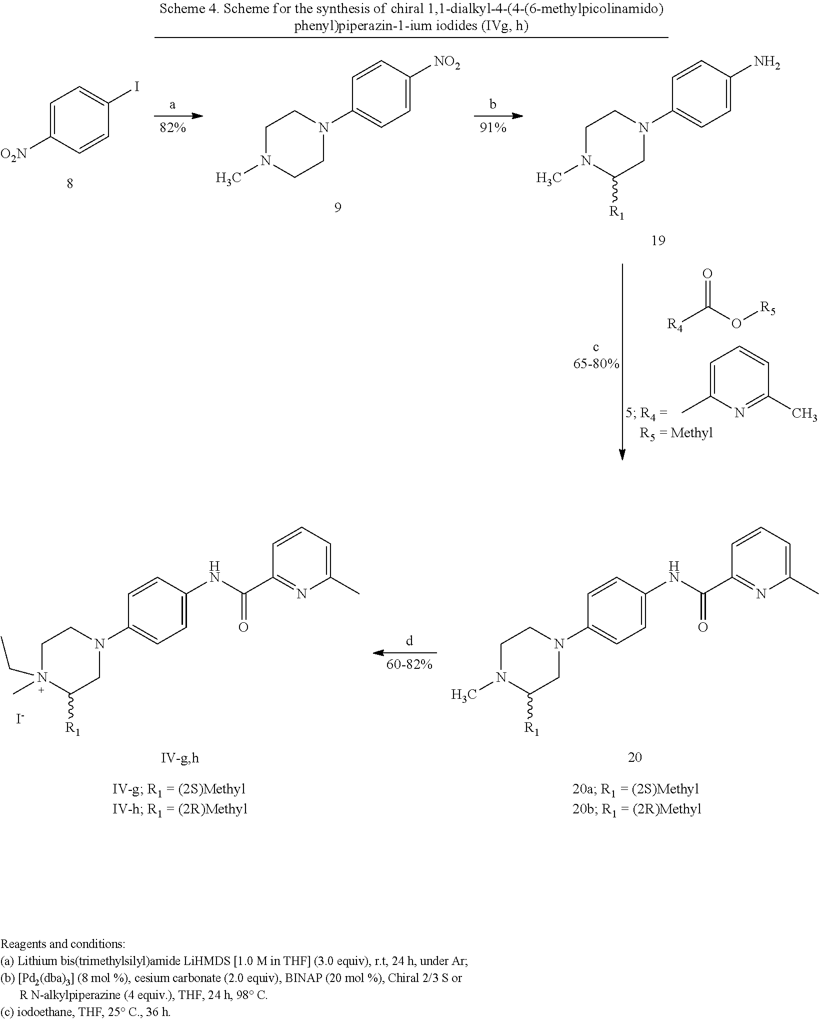

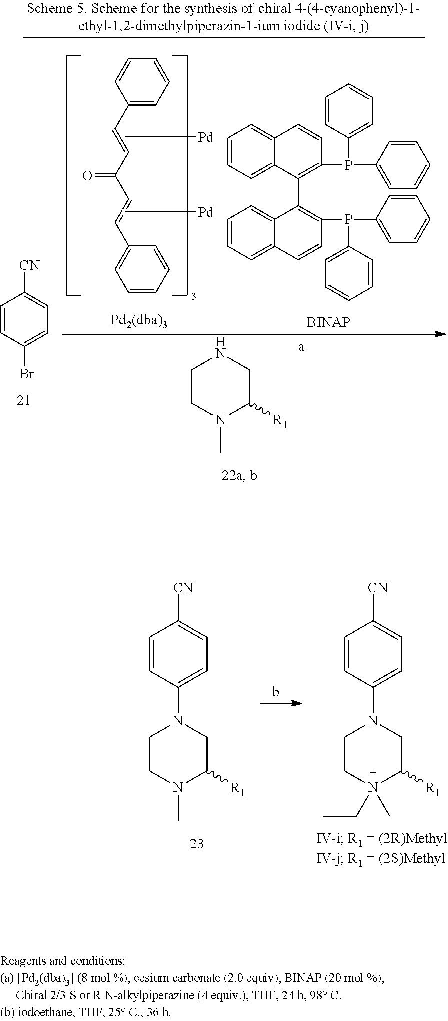

Example 3. Synthesis of 4-(4-Arylamidophenyl)-1-ethyl-1-methylpiperazin-1-ium Iodides.

The synthesis of 4-(4-arylamidophenyl)-1-ethyl-1-methylpiperazin-1-ium iodides (Va-j) is shown in Scheme 6 and carried out using the same conditions as shown in Scheme 2.

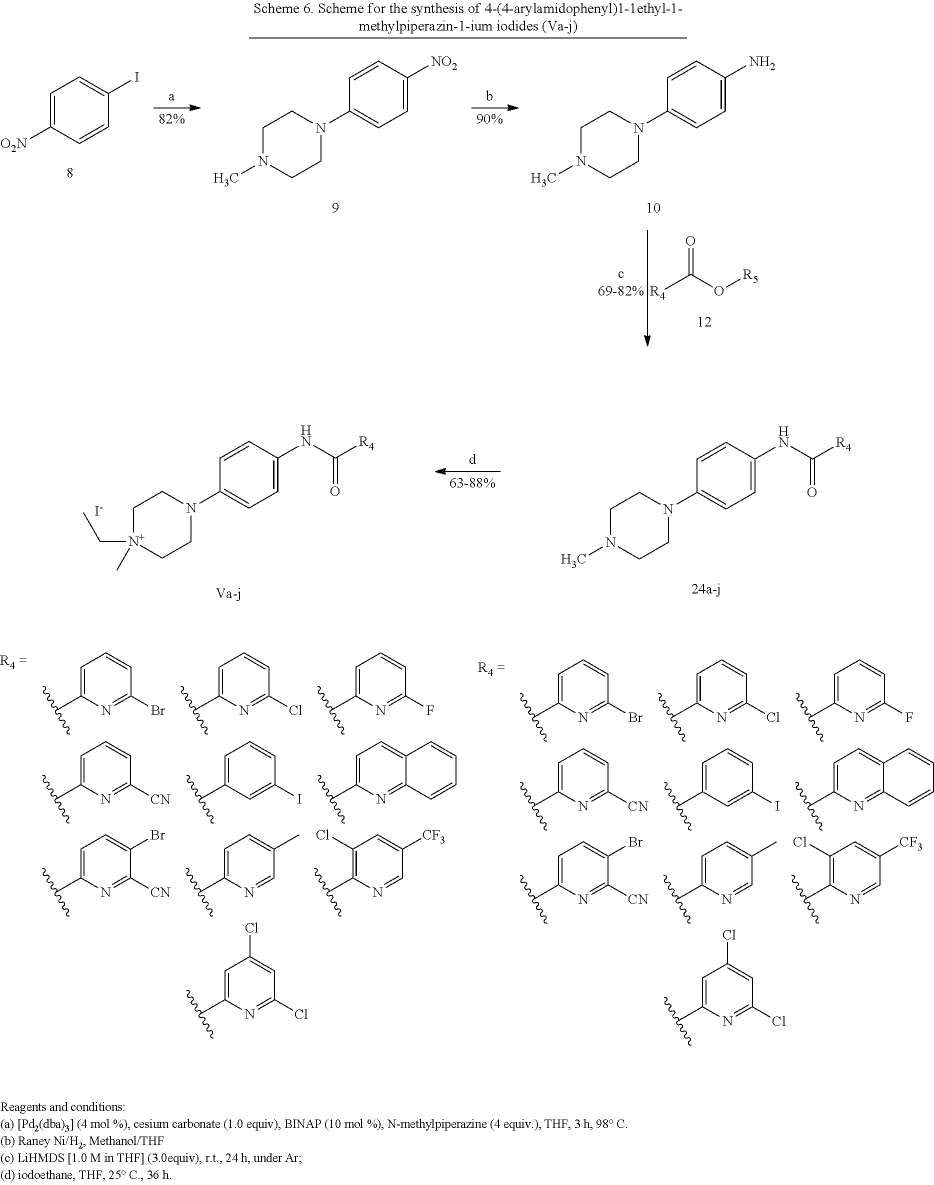

### Example 4. Synthesis of Heteroarylpicolyl Piperazine Analog.

Synthesis of analog VI by introducing oxazole with picoline is shown in Scheme 7 and was carried out using the same conditions as shown in Scheme 2.

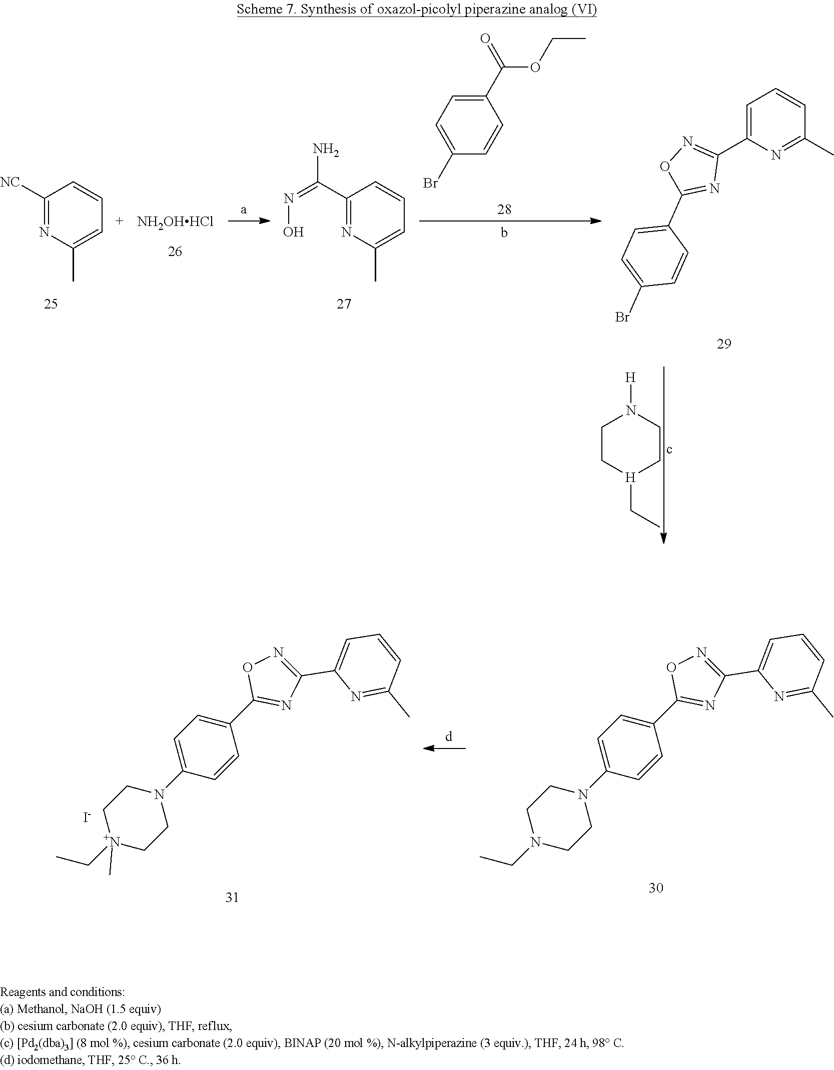

### Example 5. Molecular Docking Studies.

All docking studies were performed using glide docking by Schrodinger (Maestro version 2023-2). We utilized our recently reported homology model (template PDB: 7KOQ) for α9 for docking studies.²⁶ The protein was prepared, optimized and minimized and receptor grid was generated. Ligand_out files of ligands 3a-3l were prepared using the LigPrep, at the OPLS2005 force field and target pH (7.0±2.0) protonation state. The grid employed was sufficiently large as to encompass the orthosteric site of the receptor; grid was set to a cube of 20×20×20 Å. Using the Glide docking module of Schrodinger-Maestro 13.2, XP flexible ligand docking was performed within partial charge cutoff and the Van der Waals scaling factor was selected to be 0.15 and 0.80, respectively, for ligand atoms. Finally, the conformations with the most favorable free energy of binding were selected for analyzing the interactions between the receptor and ligands. PyMOL version 2.5.2 and Chimera 1.6 software was used for 3D molecular graphics, structural alignments and visualizations.

Example 6. Measurement of Ionic Currents of nAChR.

Plasmid DNAs encoding the human α7 and heteromeric nAChR were obtained from Jon Lindstrom (University of Pennsylvania, Philadelphia, PA). Mouse muscle subunit clones were obtained from Jim Boulter (Salk Institute, La Jolla CA) and Paul Gardner (Dartmouth, Hanover NH). The human resistance-to-cholinesterase 3 (RIC3) clone was obtained from Millet Treinin (Hebrew University, Jerusalem, Israel) and RNA co-injected with α7 to improve the level and speed of receptor expression without affecting their pharmacological properties. Plasmid DNA encoding the human α10 nAChR was obtained from J. Michael McIntosh. Plasmid DNA encoding the human α9 nAChR and the human receptor-associated protein of the synapse (RAPSYN) with codon optimization for expression in Xenopus laevis were obtained from Katrin Richter. RAPSYN RNA was co-injected with the α9 and α10 to improve expression. After linearization and purification of the plasmid DNAs, RNAs were prepared using the mMessage mMachine in vitro RNA transcription kit (Ambion, Austin, TX). Frogs were maintained in the Animal Care Service facility of the University of Florida, and all procedures were approved by the University of Florida Institutional Animal Care and Use Committee (approval number 202002669). In brief, the animals were first anesthetized for 15-20 min in 1.5 l frog tank water containing 1 g of MS-222 buffered with sodium bicarbonate. Oocytes were obtained surgically from mature female Xenopus laevis (Nasco, Ft. Atkinson WI, USA) and treated with 1.4 mg/ml type 1 collagenase (Worthington Biochemicals, Freehold NJ, USA) for 2-4 h at room temperature in Ca²⁺-free Barth's solution (88 mM NaCl, 1 mM KCl, 2.38 mM NaHCO₃, 0.82 mM MgSO₄, 15 mM HEPES, and 12 mg/l tetracycline, pH 7.6) to remove the ovarian tissue and the follicular layers. Stage V oocytes were injected with 4-6 ng CHRNA7 RNA and 2-3 ng RIC3 RNA (2:1 ratio) in 50 nl water, or with 12 ng CHRNA9 RNA and 3 ng RAPSN RNA, or along with 12 ng CHRNA10 RNA in 50 nl water. Oocytes were maintained in Barth's solution containing 0.32 mM Ca(NO₃)₂ and 0.41 mM CaCl₂, and recordings were carried out 2-20 days after injection.

Two-electrode voltage-clamp experiments were conducted using OpusXpress 6000A (Molecular Devices, Union City CA, USA). Both the voltage and current electrodes were filled with 3 M KCl. Oocytes were voltage-clamped at −60 mV at room temperature. The oocytes were perfused with Ringer's solution (115 mM NaCl, 2.5 mM KCl, 1.8 mM CaCl₂), 10 mM HEPES, 1 μM atropine, pH 7.2) at 2 ml/min. To evaluate the effects of experimental compounds, responses were compared to control ACh-evoked responses, defined as the average of two initial applications of 60 μM ACh made before test applications. Drug applications were 12 s in duration followed by 181 s washout periods.

The responses were calculated as both peak-current amplitudes and net charge, as previously described. Data were collected at 50 Hz, filtered at 20 Hz, and analyzed by Clampfit (Molecular Devices) and Excel (Microsoft, Redmond, WA, United States). Data are expressed as means±SEM from at least five oocytes for each experiment and plotted with Kaleidagraph 4.5.2 (Abelbeck Software, Reading, PA, United States). Each episode of data acquisition was a total of 210 s and included an initial 30 s period used to define the baseline for the drug-evoked responses. After 30 s, drugs were applied, and the following 120 s were defined as the drug response period for analysis. Data reported for α7 are net charge, while peak currents are used for α9 and α9/α10 responses since these receptors do not show the same concentration-dependent desensitization that invalidates peak currents as measurements of α7 concentration-dependent responses. The values for the curve fits were generated using the Levenberg-Marquardt algorithm to obtain the best Chi-Square fit to the Hill equation using the Kaleidagraph 4.5.2 plotting program. The errors in the tables are the calculated standard errors of the fit parameters based on the goodness of fit.

### Example 7. Caco-2 Cell Permeability Assay.

Caco-2 cells at a passage of 33 were seeded on 24-well Transwell® cell culture inserts (Corning Incorporated, Corning, NY, USA) with 0.4 μm pore size and surface area of 0.33 cm² and used for study after 21-25 days post-seeding. Prior to the experiment, DMEM was replaced with warm HBSS buffer (pH 7.4). The integrity of the Caco-2 monolayer was verified by measuring the transepithelial electrical resistance (TEER) value across the monolayer using Millicell, ERS meter (Millipore, Bedford, MA) and the wells with TEER value over 250 Ω·cm² were used for permeability assessment. The pH-dependent permeability of 3 h and 3f was assessed at pH 5.0, 6.8, and 7.4 at a concentration of 5 μg/mL. The pH of the basolateral compartment was kept at pH 7.4 for all experiments and all experiments were conducted in triplicate. Propranolol (5 μg/mL) and atenolol (10 μg/mL) were used as high and low permeability markers while digoxin (10 μg/mL) was used as a p-glycoprotein (p-gp) substrate to validate the assay. The apical (A) to basolateral (B) transport experiments at different pH across the Caco-2 monolayer were conducted by adding 0.1 mL of compound solution in the apical compartment of the inserts and 0.6 mL blank HBSS buffer (pH=7.4) in the basolateral compartment. The basolateral (B) to apical (A) transport experiments across the Caco-2 monolayer were conducted by adding 0.1 mL of blank HBSS buffer in the apical compartment of the inserts and 0.6 mL compound solution in the basolateral compartment. Aliquots (25 L) were collected from the basolateral and apical compartments at 0, and 2 h, and 100 μL of acetonitrile containing phenacetin as internal standard was added and vortex mixed for 2 min followed by filtration using 0.45 μm Polytetrafluoroethylene (PTFE) filter plates by centrifugation for 5 min at 2000 rpm (1500 g). The filtrates were injected into UPLC-MS/MS for quantitative analysis. Permeability marker (propranolol and atenolol) samples were also processed in a similar manner and analyzed by UPLC-MS/MS.

Data analysis: The apparent permeability coefficient (Papp) was calculated as

$${P}_{app} = ( \frac{{V}_{A}}{Area \times Time} ) \times ( \frac{{[ drug ]}_{acceptor}}{{[ drug ]}_{initial , donor}} )$$

Where [drug]_{acceptor} is the concentration of the drug in acceptor compartment and; [drug]ᵢₙᵢₜᵢₐₗ, donor is the initial concentration of drug in donor compartment; Area is the surface area of the membrane (0.33 cm²); time is the total transport time in seconds; VA is the volume (in mL) in the acceptor well. The efflux ratio was calculated using following equation:

$$Efflux Ratio ( E R ) = {P}_{app ( A \to B )} / {P}_{app ( B \to A )}$$

### Example 8. Plasma Stability of Compounds.

To know the stability of the compounds in the mice plasma, in vitro stability study was conducted using pooled BALB/c mice plasma (purchased from commercial source (BioIVT). The blank mice plasma (250 μL) was preincubated in a shaking incubator for 10 minutes prior to spiking the compound at 37° C. the compounds were spiked at a concentration of 1 μM and incubated again in a shaking incubator. Procaine was used as a positive control. A 25 μL of the plasma was sampled at 0, 5, 10, 15, 20, 30 and 60 min. Plasma proteins were precipitated by adding 100 μL of acetonitrile containing phenacetin as internal standard and vortex mixed for 2 min followed by filtration using 0.45 μm PTFE filter plates by centrifugation for 5 min at 2000 rpm. The filtrate was injected to UPLC-MS/MS for quantitative analysis.

Quantification of 3 h and 3f: Both 3 h and 3f were quantified using a Waters Acquity Class-I UPLC coupled with a Xevo TQ-S Micro triple quadrupole mass spectrometer (Milford, MA, USA). Chromatographic separation was achieved on a Waters Acquity BEH C18 column (1.7 μm, 2.1×50 mm). The mobile phases used were 0.1% formic acid in water (A) and acetonitrile (B) at a 0.35 mL/min flowrate and with the following gradient: initial condition as mobile phase A at 95% held for 0.4 min, linearly decreased to 20% reaching 1.6 min and maintained till 2.4 min, then sharply decreased back to the initial conditions by 2.5 min and maintained until 3.0 min for re-equilibration. The column and autosampler temperatures were kept at 50° C. and 4° C., respectively. The mass spectrometer was operated in positive ion mode and detection of the ions was performed in Multiple reaction monitoring (MRM) mode, monitoring the transitions of m/z 339.3 precursor ion [M+H]⁺ to the m/z 86.0 product ion for both 3 h and 3f, m/z 180.12 precursor ion [M+H]⁺ to the m/z 110.03 product ion for internal standard (phenacetin). MassLynx 4.2 was used for data acquisition and TargetLynx for data analysis. The ion spray voltage was set at 2000 V, the desolvation temperature was 400° C., the desolvation gas flow was 900 L/h, and the cone gas flow was 40 L/h.

### Example 9. Experiments Using Cultured THP-1 Cells and Human Peripheral Blood Mononuclear Cells.

The monocytic THP-1 cell line was obtained from the German Collection of Microorganisms and Cell Cultures (Braunschweig, Germany). THP-1 cells were cultured under 5% CO₂ atmosphere at 37° C. in RPMI 1640 medium (Capricorn, Ebsdorfergrund, Germany, Cat #RPMI-A) supplemented with 10% fetal bovine serum (FBS) from Capricorn (Cat #FBS-16A). For the experiments, the cells were spun down (500 g, 8 min, 19° C.) and resuspended in FBS-free RPMI 1640 medium. Thereafter, 0.5×10⁶ cells/0.5 ml and per well were seeded in 48-well plates (Greiner Bio-One, Frickenhausen, Germany) as described previously.²⁷ The cells were primed with LPS (1 μg/ml; E. coli O26:B6, Merck, Darmstadt, Germany, Cat #L2654) for 5 h under 5% CO2 atmosphere at 37° C. Thereafter, the P2X7 receptor agonist BzATP (100 μM; Jena Bioscience, Jena, Germany, Cat #NU-1620-5) was added for 40 min in the absence or presence of the compounds 2, 3f and 3h or the cholinergic agonist ACh (10 μM; Merck, Cat #A6625). After the treatment, cells were spun down (500 g, 8 min, 4° C.) to collect the cell-free supernatants, that were stored at −20° C. for later measurements of IL-1β and lactate dehydrogenase (LDH) activity.

Human PBMCs were isolated from blood samples obtained from healthy (self-reported) female and male non-smoking adult volunteers. The study was approved by the ethics committee of the medical faculty Giessen, Germany (No. 90/18) and performed in accordance with the Helsinki Declaration. PBMC isolation was performed using Leucosep gradients (Greiner Bio-One, Cat #227288) as described previously.²⁷ To prime the cells with LPS, 5 ng/ml LPS was added to blood samples before the gradient centrifugation. After the isolation process, 0.5×10⁶ cells/0.5 ml and well were seeded in 48-well plates (Greiner Bio-One) in Monocyte Attachment Medium (PromoCell, Heidelberg, Germany, Cat #C-28051) for 3 h. Non-adherent cells were removed, and cell culture medium was replaced by fresh RPMI 1640 medium (Sigma-Aldrich, Cat #R8758). Stimulation with BzATP in the presence or absence of the compounds 2, 3f or ACh was done as described for THP-1 cells.

To measure IL-1β concentrations in the cell-free supernatants the Human IL-1 beta/IL-1F2 DuoSet enzyme-linked immunosorbent assay (ELISA) from R&D Systems (Cat #DY201) was used according to the supplier's instructions. In parallel, LDH activity was measured to test for cell viability at the end of the cell culture experiments, using the CytoTox 96® Non-Radioactive Cytotoxicity Assay (Promega, Madison, WI, United States; Cat #G1780) according to the supplier's instructions. The viability of the cells was not impaired in any condition tested (data not shown).

Results obtained in the BzATP-induced IL-1β release experiments were analyzed using SPSS (Version 27, IBM, Armonk, NY, United States). The data were analyzed first by the Friedman test followed by the Wilcoxon signed-rank test. Data were visualized using Inkscape version 0.48.5 r10040 (Free and Open Source Software licensed under the GPL). The number (n) of individual experiments is indicated in the Figures and refers to independent experiments, which were performed on different days with different cell passages.

### Example 10. Measurement of Inflammatory Pain.

Experiments were conducted using adult (10-15 weeks) male and female C57BL/6J mice from Jackson Laboratory (Bar Harbor ME, USA): α7 WT and KO mice on a C57BL/6J background. Mice null for the α7 subunit along with their WT littermates were initially procured from Jackson Laboratory and later bred in an approved animal care facility at Virginia Commonwealth University. The breeding scheme involved crossing heterozygous mice and backcrossing progeny for at least 12 to 15 generations, to control for irregularities that might occur crossing solely mutant animals, to generate both mutant and WT animals. Then mice were weaned at 21 days of age and subsequently housed in groups of two to five with Teklad corn cob bedding (#7097, Envigo Teklad, Madison WI, USA). Initially, they were maintained in a temperature- and humidity-controlled vivarium space (21±3° C., 55±10%) on a 12-h light/dark cycle (lights on at 7:00 AM) with free access to food (Teklad LM-485 mouse sterilized diet, Harlan Laboratories Inc., Indianapolis IN, USA) and water until needed. Then mice were retrieved from the vivarium and housed (4-5 mice per cage) for the duration of the study in a temperature- and humidity-controlled out-of-vivarium space on the same light/dark cycle. They were given ad libitum food and water. All experiments were performed during the light cycle. This study was approved by the Institutional Animal Care and Use Committee of Virginia Commonwealth University (approval #AM10142) and carried out in accordance with the National Institutes of Health's Guide for the Care and Use of Laboratory Animals. All experimental animals were included in further behavioral testing and none of them showed behavioral disturbances unrelated to the pain induction procedure.

### Induction of Inflammatory Pain by Complete Freund's Adjuvant (CFA)

The effects of 3 h and 3f were explored using the CFA test, composed of inactivated and dried Mycobacterium tuberculosis and adjuvant, a widely used model of persistent inflammatory pain. CFA was purchased from Sigma-Aldrich (St. Louis MO, USA). The CFA model is based on hypersensitivity, paw swelling, and nuclear factor-κB-mediated transcription of tumor necrosis factor α involved in the formation of the principal mediators of inflammation. Mice were injected intraplantarly with 20 μl of CFA (50%, diluted in mineral oil; Sigma-Aldrich). Mechanical sensitivity (see the measurement of the von Frey test) was measured before and 3 days after CFA injection. Compound 3h (2 and 10 mg/kg body weight (bw)) and 3f (20 mg/kg bw), dissolved in a mixture of 1:1:18 [1 volume ethanol/1 volume Emulphor-620 (Rhone-Poulenc, Inc., Princeton NJ, USA)/18 volumes distilled water] or vehicle was injected intraperitoneally (i.p.) on day 3 after CFA injection, and mice were tested for mechanical sensitivity at different time points (1, 3, 6, 24 and 72 h and 5 days) for compound 3 h and 1, 3, 6, 24 and 72 h for compound 3f after drug injection.

### Evaluation of Mechanical Sensitivity

A series of calibrated von Frey filaments (Stoelting, Wood Dale IL, USA) with logarithmically incremental stiffness ranging from 2.83 to 5.07 expressed as diameter sensitivity (ds) log 10 of 10× force (in milligrams) was applied to the paw with a modified up-down method. The mechanical threshold was expressed as log 10 of 10× force (in milligrams), indicating the force of the von Frey hair to which the animal reacted (paw withdrawn, licking, or shaking). All behavioral testing on animals was performed in a blinded manner.

The data obtained were discrete values that were not normally distributed and, hence, not suitable for parametric analysis. The data were therefore first evaluated with the non-parametric Kruskal-Wallis test, a one-way analysis of variance by ranks. A significant Kruskal-Wallis test indicated that at least one sample stochastically dominated the other samples, in this case, the baseline data for all groups. For analyzing the specific sample pairs at the different time points for stochastic dominance, as a second step the groups were tested pairwise with the Mann-Whitney rank sum test. The p values calculated by the Mann-Whitney rank sum test are provided in the tables 1a-h or shown in the FIGS. 11 and 12.

### Locomotor Activity

Mice were placed into individual Omnitech photocell activity cages (28×16.5 cm) (Columbus OH, USA) 6 h after administration of either vehicle or compound 3h (10 mg/kg bw, i.p.). Interruptions of the photocell beams (two banks of eight cells each) were then recorded for the next 60 min. Data are expressed as number of photocell interruptions.

### Example 11. Synthesis of Oxodiazole and Thiophene Containing Alpha9 Agonists

### Synthesis of Novel Oxazole-Based Alpha9 Agonist Analogs

The synthetic approach used for the synthesis of N,N-dialkyl-4-(methyl pyridine) oxadiazole)phenyl)piperazinium iodides (compounds 0 and 4) is depicted below. The formation of an amidoxime 1 was afforded through the nucleophilic attack of hydroxylamine hydrochloride (NH2OH HCl) (2.0 equiv) on a nitrile containing molecule 1a (1.0 equiv) and sodium hydroxide (NaOH) as a base (2.0 equiv). This initial stage in this multistep synthesis proceeded with excellent yields (94.6%) at room temperature (Table 6). Once the amidoxime 1 formed, the next step was the oxadiazole formation. The oxadiazole was formed with the nucleophilic attack by the amidoxime (1.0 equiv) on the aryl ester (Ethyl 4-bromobenzoate, 2a, 1.5 equiv), leading to cyclization and dehydration to afford compound 2 whilst in the presence of cesium carbonate (2.0 equiv) as a base and tetrahydrofuran (THF) as the solvent. This reaction was completed within 24 hours while being stirred at 90° C. in an oil bath. This reaction produced an overall yield of roughly 39% (Table 6), which can be attributed to the steric hindrance present within the bulky molecule, leading to lower conversion yields. The product that was obtained was carried forward to perform Buchwald-Hartwig C—N cross-coupling reactions. The reaction conditions to afford compound 3 was carried out using 1-ethyl piperazine (3a) (3.0 equiv), tris(dibenzylideneacetone)dipalladium⁰ (Pd₂(DBA)₃) (8-10 mol %) as the catalyst, cesium carbonate (2.0 equiv) as a base, ligand BINAP (10 mol %) in THF. The reaction was stirred at 90-100° C. in an oil bath for 24-48 hours to obtain roughly 14% conversion yields (Table 6). Purification of 3 was completed using reverse-phase column chromatography given the high polarity of 3. The Buchwald-Hartwig C—N cross coupling step became the bottleneck of this multistep synthesis. Thus, post purification, the overall yields were low which warranted us to experiment with differing reaction conditions. We decided to select a different ligand (VPhos, 5 mol %) and LiHMDS (3.3 equiv) as a base, while retaining the same catalyst, solvent and stirring the reaction mixture at 70° C. in an oil bath. However with these reaction conditions, there was little to no product formation. With this outcome, we decided to alter the heating source by switching from oil baths to microwaved reactions at 100° C.

TABLE 6
Showcase of all intermediate and final compounds synthesized in the oxazole-based Alpha9 agonist analogs.

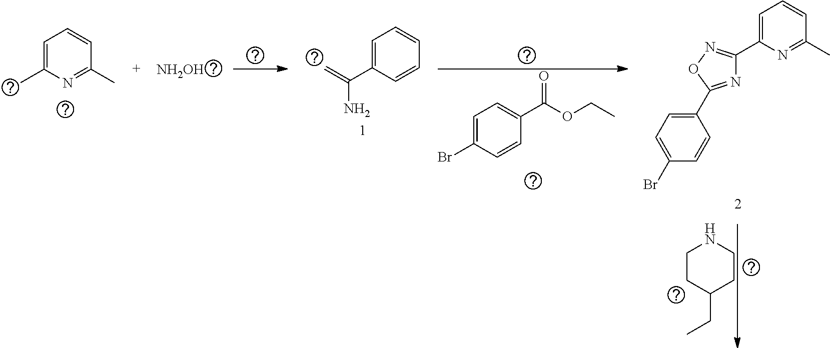

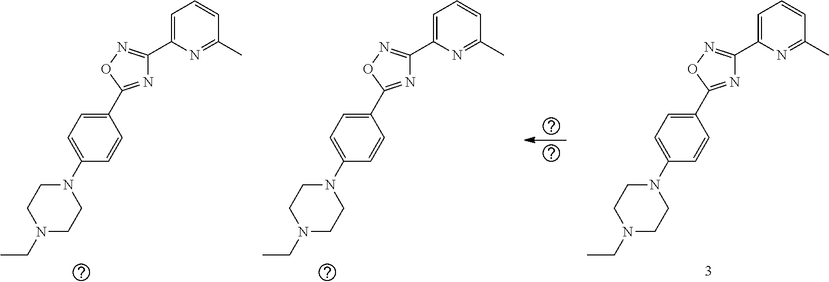 [SMILES: CCN1CCN(c2ccc(-c3nc(-c4cccc(C)n4)no3)cc2)CC1 ; CCN1CCN(c2ccc(-c3nc(-c4cccc(C)n4)no3)cc2)CC1 ; CCN1CCN(c2ccc(-c3nc(-c4cccc(C)n4)no3)cc2)CC1]

| Compd. | | Amsonni | | |
|---|---|---|---|---|
| No. | Structures | Obtained | Yield | Purity |
| 1 | 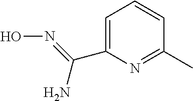 | 1.21g | 94.60% | [symbol] |
| 2 | 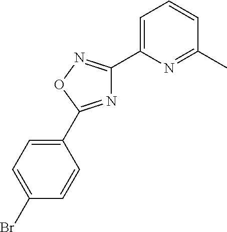 [SMILES: Cc1cccc(-c2noc(-c3ccc(Br)cc3)n2)n1] | 0.8g | 38.60% | [symbol] |
| 3 | 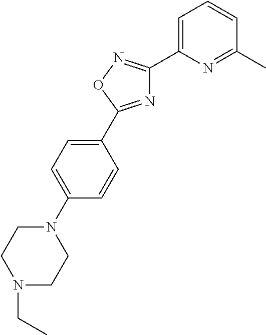 [SMILES: CCN1CCN(c2ccc(-c3nc(-c4cccc(C)n4)no3)cc2)CC1] | 0.062g | 14% | [symbol] |
| 4 | 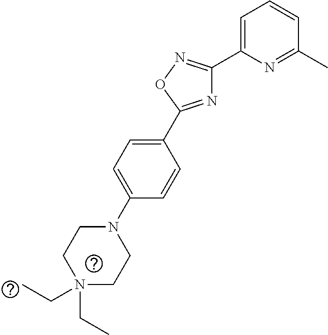 | 0.020g | 66.70% | 98% |
| D | 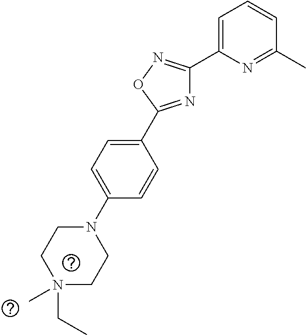 | 0.025g | 86.20% | 96% |

[symbol]

indicates data missing or illegible when filed

### Synthesis of Novel Thiophene-Based Alpha9 Agonist Analogs

The synthetic approach used for the synthesis of N,N-dialkyl-4-(methyl pyridine)carbamoyl)thiophene)piperazinium iodides (compounds 7, 8, 22, and 23) is depicted below. The formation of compounds 5 and 20 was achieved through amidation of the primary amine (5b and 20b, 1.5 equiv) and carboxylic acid (5a and 20a, 1 equiv) using Hexafluorophosphate Azabenzotriazole Tetramethyl Uronium (HATU) (1 equiv) as the coupling agent. N,N-Diisopropylethylamine (DIPEA) (4.0 equiv) was used as a base, dichloromethane (DCM) was the solvent and the reaction was stirred overnight (24 hrs) at room temperature. This initial stage in this multistep synthesis proceeded with exceptional yields of 84% and 89% for compounds 5 and 20 respectively (Table 7). The next step in this synthesis was the Buchwald-Hartwig C—N cross coupling reaction using the same traditional reaction conditions discussed in the oxadiazole-based analogs synthesis. As anticipated, the overall yields of these reactions to afford compounds 6 and 21 were low (11.4% and 24% respectively) but enough to carry forward in the last step of synthesis. Compound 6 was treated with iodomethane and iodoethane to obtain final compounds 7 (with a yield of 55.6% and purity of 95%) and 8 (with a yield of 35.7% and purity of 96%) respectively (Table 7). Final compounds 22 and 23 were similarly afforded by treating compound 21 with iodomethane and iodoethane respectively. Compound 22 was obtained with a yield of 21.4% and purity of 98%. Compound 23 was obtained with a yield of 32.9% and purity of 99% (Table 7).

TABLE 7
Showcase of all intermediate and final compounds synthesized in the thiophene-based alpha9 agonist analogs.

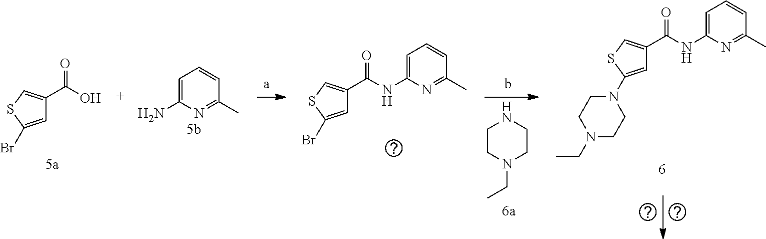

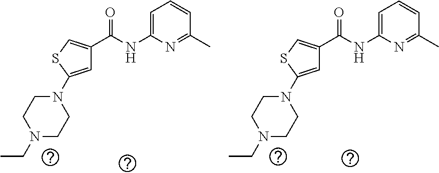 [SMILES: CCN1CCN(c2cc(C(=O)Nc3cccc(C)n3)cs2)CC1 ; CCN1CCN(c2cc(C(=O)Nc3cccc(C)n3)cs2)CC1]

| Compd. | | Amount | | |
|---|---|---|---|---|
| No. | Structures | Obtained | Yield | Purity |
| 5 | 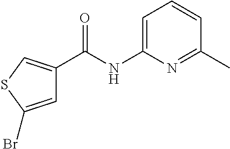 [SMILES: Cc1cccc(NC(=O)c2csc(Br)c2)n1] | 0.6g | 84% | [symbol] |
| 6 | 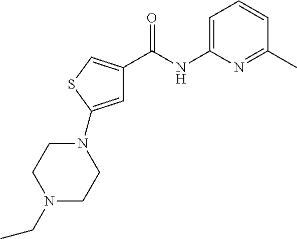 [SMILES: CCN1CCN(c2cc(C(=O)Nc3cccc(C)n3)cs2)CC1] | 0.084g | 11.40% | [symbol] |
| 7 | 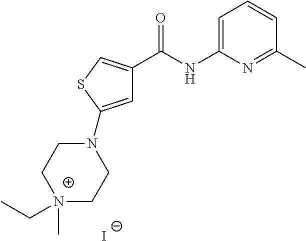 [SMILES: CC[N+]1(C)CCN(c2cc(C(=O)Nc3cccc(C)n3)cs2)CC1 ; [I-]] | 0.015g | 55.50% | 95% |
| 8 | 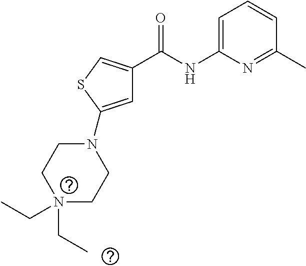 | [symbol] | 35.70% | 96% |
| 20 | 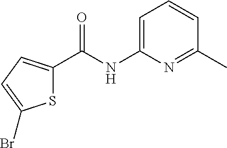 [SMILES: Cc1cccc(NC(=O)c2ccc(Br)s2)n1] | 2.55g | 89% | [symbol] |
| 21 | 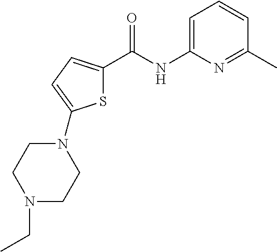 [SMILES: CCN1CCN(c2ccc(C(=O)Nc3cccc(C)n3)s2)CC1] | 0.134g | 24% | [symbol] |
| 22 | 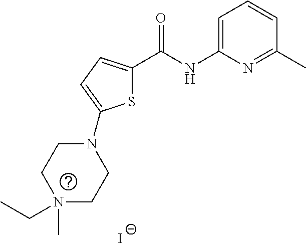 | 0.015g | 21.40% | 98% |
| 23 | 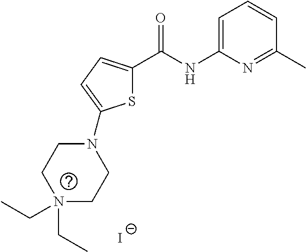 | 0.024g | 32.90% | 99% |

[symbol]

indicates data missing or illegible when filed

Example 12. Synthesis of 4-(3-(6-(halide substituted)picolinamido)phenyl)-1-ethyl-1-methyl Piperazin-1-ium-2-ide Iodides.

The general approach for the formation of 4-(3-(6-(halide substituted) picolinamido)phenyl)-1-ethyl-1-methyl-Piperazin-1-ium-2-ide Iodides (1a-c) and 1,1-Dialkyl-4-(substituted aryl/alkyl)carbamoyl)phenyl)piperazinium Iodides (2a-d) are detailed in the two schemes shown below.

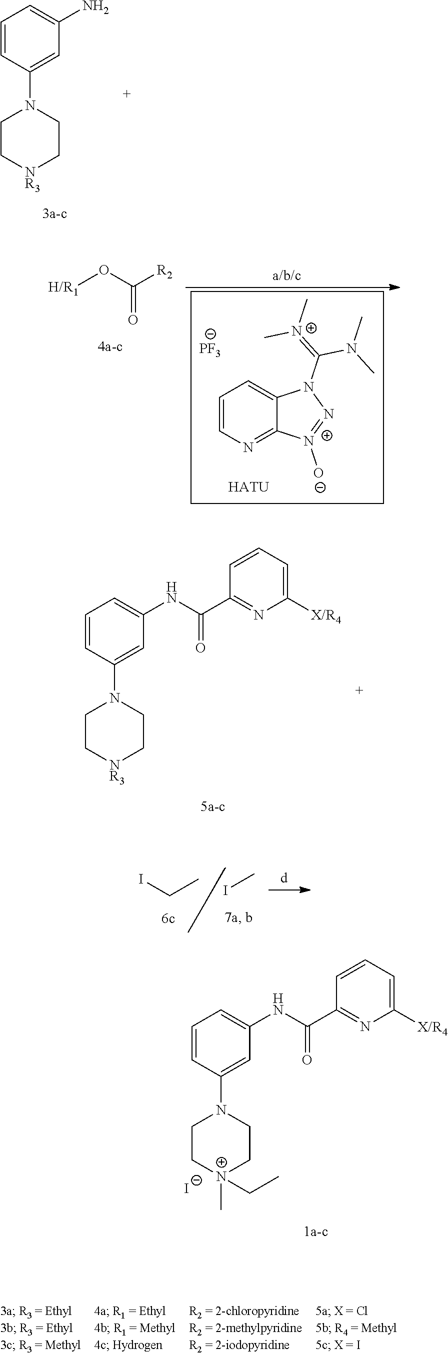

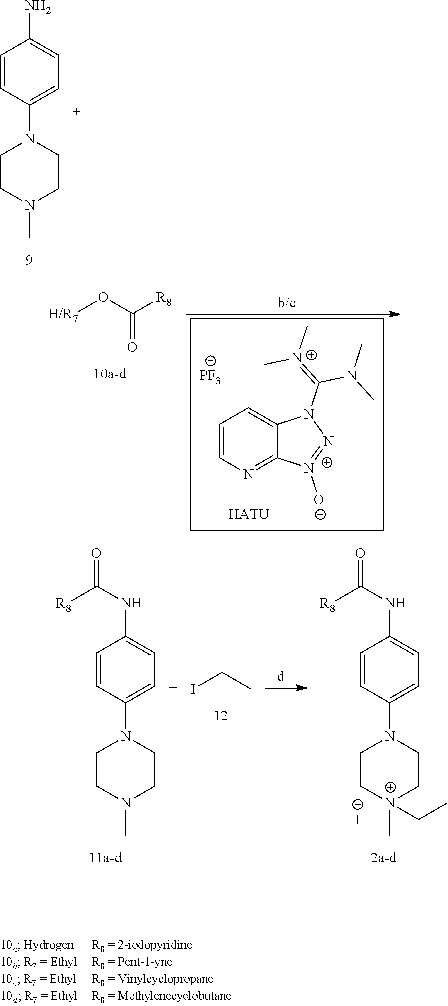

Amidation of 3-(4-ethylpiperazin-1-yl) aniline (3a,b) and 3-(4-methylpiperazin-1-yl) aniline (3c) (Scheme 1) formed N-(3-(4-(methyl/ethyl)piperazin-1-yl)phenyl)picolinamides (5a-c); these reactions, with conditions a, b, and c in scheme 1 preceded with 18%, 89%, and 80% yields respectively at room temperature. Reaction conditions (a) and (b) were done with a metal free amidation using ethyl/methyl picolinates at 1.2 and 1.1 eqv's respectively. In reaction condition (a) Sodium bis(trimethylsilyl)amide (NaHMDS) [1M in THF] was used in 2.0 eqv as a base, and reaction condition (b) was done with Lithium bis(trimethylsilyl)amide (LIHMDS) [1M in THF] in 3.0 eqv as a base, and tetrahydrofuran was used as a solvent in the presence of argon for both (a) and (b) conditions. Reaction condition (c) was also a metal free amidation with a picolinic acid at 1.1 eqv. This reaction condition used Hexafluorophosphate Azabenzotriazole Tetramethyl Uronium (HATU) at 1.0 eqv as a coupling reagent along with N,N-Diiso propylethylamine (DIPEA), Hunig's Base, at 4.0 eqv, as a base, in dichloromethane as a solvent at room temperature. These three different reagent combinations were used to couple the picolinic group into an amide bond with (3a-c). Reaction conditions (b) and then (c) produced the highest yield. Condition (b) ran for 48 hours, however, it was noted that the reaction was 95% complete after 24 hours but was left to run longer for complete conversion. This leaves condition (b) as the most effective for this step in Scheme 1. Once the N-(3-(4-(methyl/ethyl)piperazin-1-yl)phenyl)picolinamides (5a-c) were formed they were converted to quaternary ammonium methyl-ethyl salts by either methyl iodide (7a,b) in 5eqv for 90 minutes or by ethyl iodide (7c) in 10eqv for 24 hours, both in tetrahydrofuran as a solvent and under argon. These were purified by recrystallization to give products 1a-c, all formed with 90% yield.

Amidation of 4-(4-methylpiperazin-1-yl) aniline (9a-d) formed N-(4-(4-methylpiperazin-1-yl)phenyl)amides (11a-d); 11a (condition c) formed with 51.3% yield, 11b (condition b) with 81.7% yield, 11c (condition b) with 42.5% yield, and 11d (condition b) with 62.3% yield, all in room temperature. Reaction conditions (b) and (c) were performed in the same manner as in scheme 1. Once the N-(4-(4-methylpiperazin-1-yl)phenyl)amides (11a-d) were formed they were converted to quaternary ammonium methyl-ethyl salts (2a-d) by ethyl iodide (12) in 10eqv for 48 hours and purified by recrystallization. These were formed with 80-95% yield (2a,b,d), except for 2c which formed with 50% yield.

The products formed are shown below.

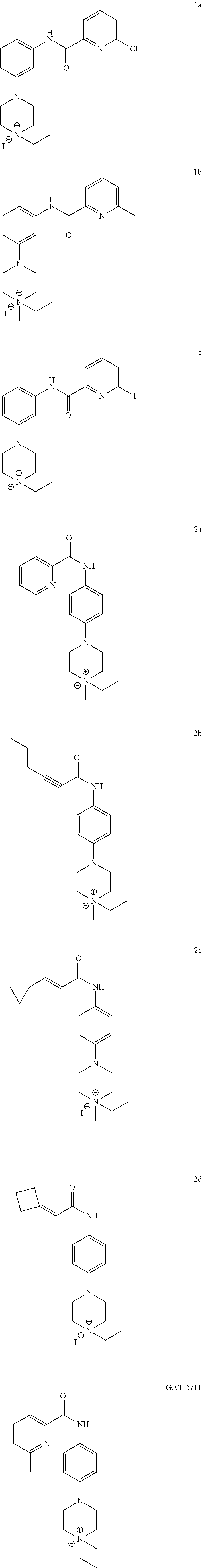

As used herein, “consisting essentially of” allows the inclusion of materials or steps that do not materially affect the basic and novel characteristics of the claim. Any recitation in the original claims of the term “comprising”, particularly in a listing of components of a composition or elements of a device, also constitutes disclosure of alternative embodiments in which “comprising” is replaced with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### REFERENCES

1. Hogg, R.; Raggenbass, M.; Bertrand, D., Nicotinic acetylcholine receptors: from structure to brain function. Reviews of physiology, biochemistry and pharmacology 2003, 1-46.
2. Halder, N.; Lal, G., Cholinergic system and its therapeutic importance in inflammation and autoimmunity. Frontiers in Immunology 2021, 12, 660342.
3. Dani, J. A.; Bertrand, D., Nicotinic acetylcholine receptors and nicotinic cholinergic mechanisms of the central nervous system. Annu. Rev. Pharmacol. Toxicol. 2007, 47, 699-729.
4. Millar, N. S.; Gotti, C., Diversity of vertebrate nicotinic acetylcholine receptors. Neuropharmacology 2009, 56 (1), 237-246.
5. Gotti, C.; Moretti, M.; Bohr, I.; Ziabreva, I.; Vailati, S.; Longhi, R.; Riganti, L.; Gaimarri, A.; McKeith, I. G.; Perry, R. H., Selective nicotinic acetylcholine receptor subunit deficits identified in Alzheimer's disease, Parkinson's disease and dementia with Lewy bodies by immunoprecipitation. Neurobiology of disease 2006, 23 (2), 481-489.
6. Takeuchi, H.; Yanagida, T.; Inden, M.; Takata, K.; Kitamura, Y.; Yamakawa, K.; Sawada, H.; Izumi, Y.; Yamamoto, N.; Kihara, T., Nicotinic receptor stimulation protects nigral dopaminergic neurons in rotenone-induced Parkinson's disease models. Journal of neuroscience research 2009, 87 (2), 576-585.
7. Mohammadi, S. A.; Christie, M. J., Conotoxin interactions with α9α10-nAChRs: Is the α9α10-nicotinic acetylcholine receptor an important therapeutic target for pain management? Toxins 2015, 7 (10), 3916-3932.
8. Quik, M.; Bordia, T.; Huang, L.; Perez, X., Targeting nicotinic receptors for Parkinson's disease therapy. CNS & Neurological Disorders-Drug Targets (Formerly Current Drug Targets-CNS & Neurological Disorders) 2011, 10 (6), 651-658.
9. Gu, S.; Knowland, D.; Matta, J. A.; O'Carroll, M. L.; Davini, W. B.; Dhara, M.; Kweon, H.-J.; Bredt, D. S., Hair cell α9α10 nicotinic acetylcholine receptor functional expression regulated by ligand binding and deafness gene products. Proceedings of the National Academy of Sciences 2020, 117 (39), 24534-24544.
10. Piovesana, R.; Salazar Intriago, M. S.; Dini, L.; Tata, A. M., Cholinergic modulation of neuroinflammation: focus on α7 nicotinic receptor. International Journal of Molecular Sciences 2021, 22 (9), 4912.
11. Reale, M.; Costantini, E., Cholinergic modulation of the immune system in neuroinflammatory diseases. Diseases 2021, 9 (2), 29.
12. Papke, R. L.; Lindstrom, J. M., Nicotinic acetylcholine receptors: Conventional and unconventional ligands and signaling. Neuropharmacology 2020, 168, 108021.
13. Bagdas, D.; Gurun, M. S.; Flood, P.; Papke, R. L.; Damaj, M. I., New insights on neuronal nicotinic acetylcholine receptors as targets for pain and inflammation: a focus on α7 nAChRs. Current neuropharmacology 2018, 16 (4), 415-425.
14. Toma, W.; Ulker, E.; Alqasem, M.; AlSharari, S. D.; McIntosh, J. M.; Damaj, M. I., Behavioral and molecular basis of cholinergic modulation of pain: focus on nicotinic acetylcholine receptors. Behavioral Pharmacology of the Cholinergic System 2020, 153-166.
15. Hone, A. J.; Servent, D.; McIntosh, J. M., α9-containing nicotinic acetylcholine receptors and the modulation of pain. British journal of pharmacology 2018, 175 (11), 1915-1927.
16. Romero, H. K.; Christensen, S. B.; Di Cesare Mannelli, L.; Gajewiak, J.; Ramachandra, R.; Elmslie, K. S.; Vetter, D. E.; Ghelardini, C.; ladonato, S. P.; Mercado, J. L., Inhibition of α9α10 nicotinic acetylcholine receptors prevents chemotherapy-induced neuropathic pain. Proceedings of the National Academy of Sciences 2017, 114 (10), E1825-E1832.
17. Elgoyhen, A. B.; Katz, E., The efferent medial olivocochlear-hair cell synapse. Journal of Physiology-Paris 2012, 106 (1-2), 47-56.
18. Elgoyhen, A. B.; Katz, E.; Fuchs, P. A., The nicotinic receptor of cochlear hair cells: a possible pharmacotherapeutic target? Biochemical pharmacology 2009, 78 (7), 712-719.
19. Plazas, P. V.; Katz, E.; Gomez-Casati, M. E.; Bouzat, C.; Elgoyhen, A. B., Stoichiometry of the α9α10 nicotinic cholinergic receptor. Journal of Neuroscience 2005, 25 (47), 10905-10912.
20. Elgoyhen, A. B.; Vetter, D. E.; Katz, E.; Rothlin, C. V.; Heinemann, S. F.; Boulter, J., α10: a determinant of nicotinic cholinergic receptor function in mammalian vestibular and cochlear mechanosensory hair cells. Proceedings of the National Academy of Sciences 2001, 98 (6), 3501-3506.
21. Verbitsky, M.; Rothlin, C. V.; Katz, E.; Elgoyhen, A. B., Mixed nicotinic-muscarinic properties of the alpha9 nicotinic cholinergic receptor. Neuropharmacology 2000, 39 (13), 2515-24.
22. Lipovsek, M.; Fierro, A.; Perez, E. G.; Boffi, J. C.; Millar, N. S.; Fuchs, P. A.; Katz, E.; Elgoyhen, A. B., Tracking the molecular evolution of calcium permeability in a nicotinic acetylcholine receptor. Molecular biology and evolution 2014, 31 (12), 3250-3265.
23. Callaghan, B.; Adams, D. J., Analgesic α-conotoxins Vc1.1 and Rg1A inhibit N-type calcium channels in sensory neurons of α9 nicotinic receptor knockout mice. Channels 2010, 4 (1), 51-54.
24. Lips, K.; Pfeil, U.; Kummer, W., Coexpression of α9 and α10 nicotinic acetylcholine receptors in rat dorsal root ganglion neurons. Neuroscience 2002, 115 (1), 1-5.
25. Ellison, M.; Haberlandt, C.; Gomez-Casati, M. E.; Watkins, M.; Elgoyhen, A. B.; Mcintosh, J. M.; Olivera, B. M., α-RgIA: A novel conotoxin that specifically and potently blocks the α9α10 nAChR. Biochemistry 2006, 45 (5), 1511-1517.
26. Papke, R. L.; Andleeb, H.; Stokes, C.; Quadri, M.; Horenstein, N. A., Selective agonists and antagonists of α9 versus α7 nicotinic acetylcholine receptors. ACS chemical neuroscience 2022, 13 (5), 624-637.

What is claimed is:

1. A compound of Formula I, wherein the compound binds to a nicotinic acetylcholine receptor comprising an alpha9, alpha10 and/or alpha7 subunit: 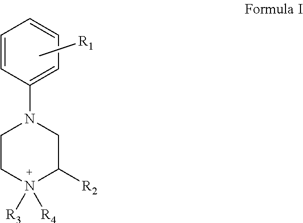
wherein R₁ is selected from the group consisting of cyano or —R₅—R₆; wherein R₅ is —CONH—, —NHCO—, oxazole, or oxadiazole; wherein R6 is C1-C6 alkyl, C1-C6 haloalkyl or dihaloalkyl, halopyridyl, cyanopyridyl, C1-C6 alkylpyridyl, optionally further halo substituted, 1-pentynyl, ethenylcyclopropyl, methenylcyclobutyl, or 2-quinolyl;
wherein R2 is hydrogen or C1-C6 alkyl; wherein R2 can be bound to any carbon on the piperazine ring and with either R or S stereochemistry;
wherein R3 and R4 are independently C1-C6 alkyl or hydrogen, with the proviso that if one of R3 and R4 is hydrogen, the other is not hydrogen, and optionally wherein R3 and R4 are fused to form a 4-, 5-, 6-, or 7-membered ring, saturated or unsaturated, optionally containing 1 or 2 heteroatoms selected from N, O, and S, and optionally substituted; and
wherein all substitutions on 6-membered rings can be ortho, meta, or para.

2. The compound of claim 1, wherein the compound is an agonist, partial agonist, or silent agonist of said nicotinic acetylcholine receptor.

3. The compound of claim 1, wherein the compound is an antagonist of said nicotinic acetylcholine receptor.

4. The compound of claim 1, wherein the compound is selected from group consisting of the following compounds:

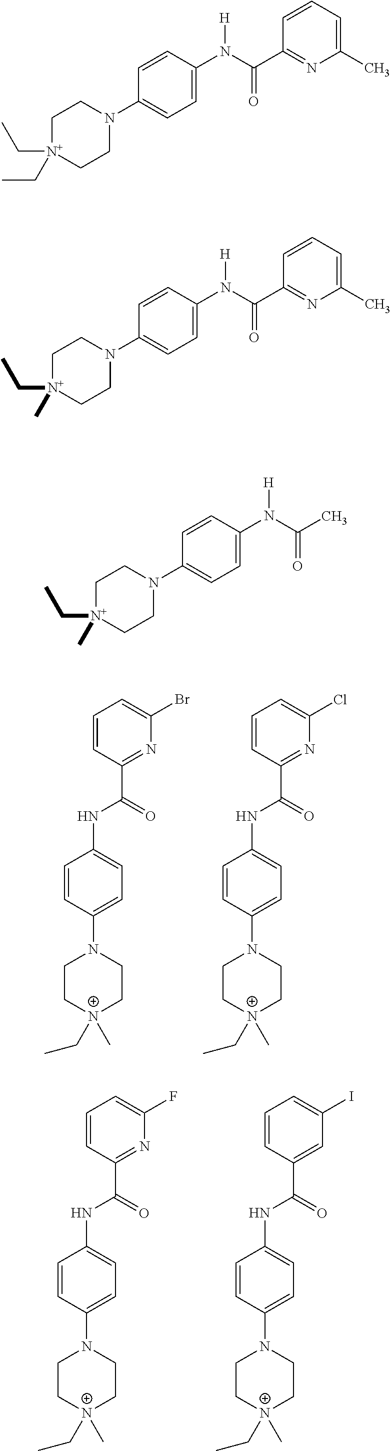

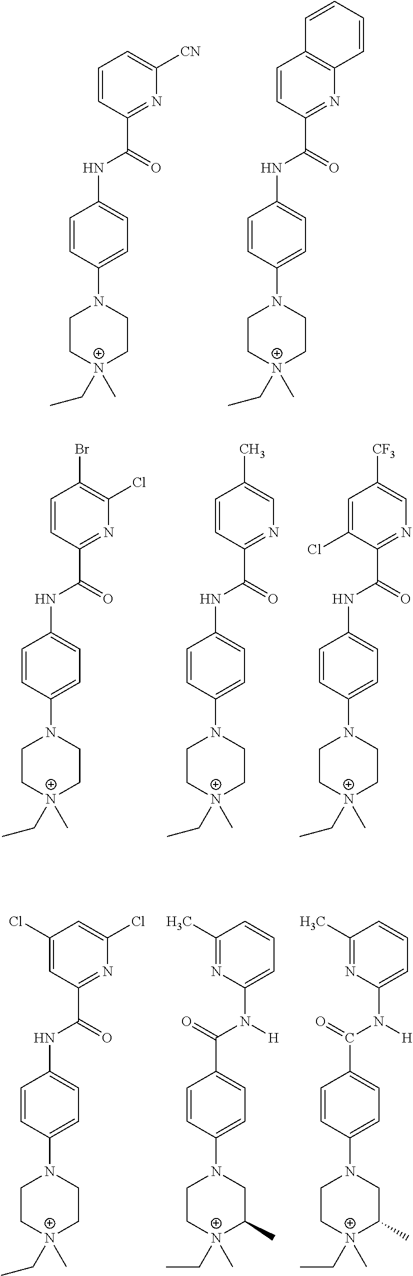

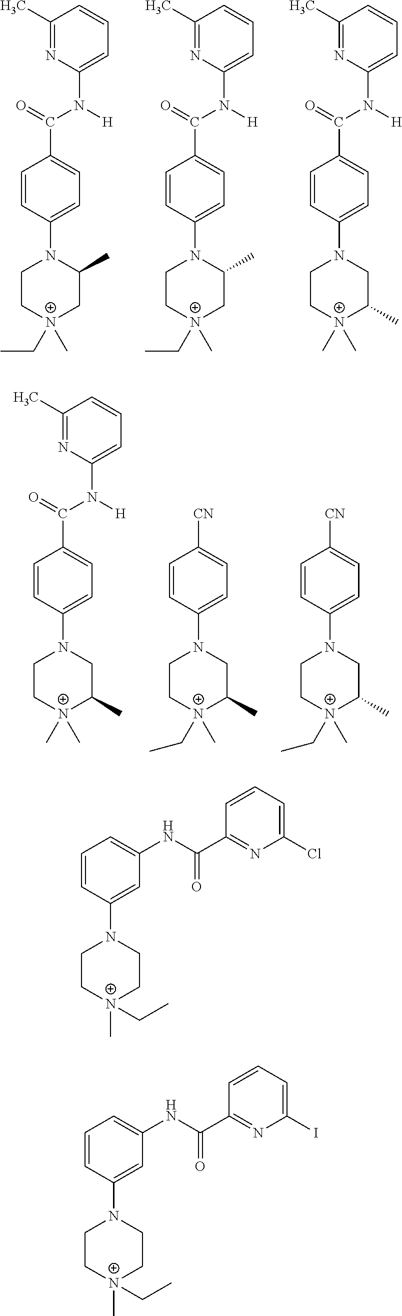

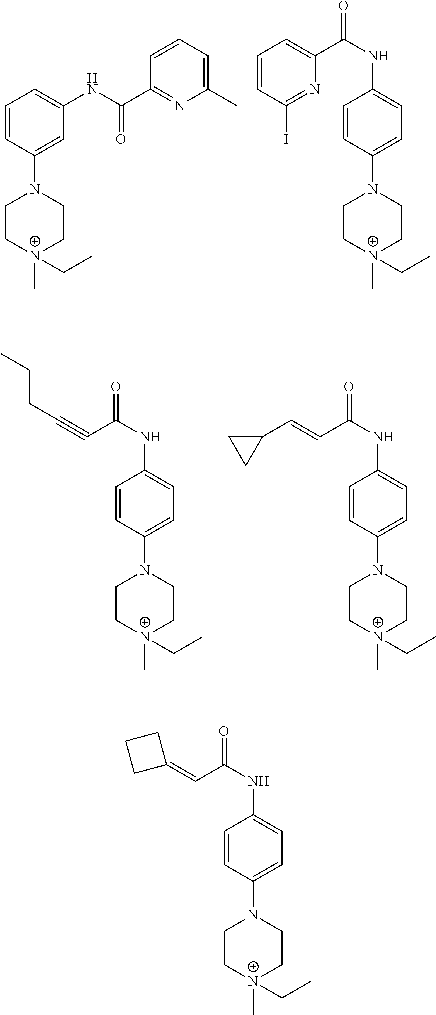 [SMILES: CC[N+]1(C)CCN(c2cccc(NC(=O)c3cccc(C)n3)c2)CC1 ; CC[N+]1(C)CCN(c2ccc(NC(=O)c3cccc(I)n3)cc2)CC1 ; CCCC#CC(=O)Nc1ccc(N2CC[N+](C)(CC)CC2)cc1 ; CC[N+]1(C)CCN(c2ccc(NC(=O)/C=C/C3CC3)cc2)CC1 ; CC[N+]1(C)CCN(c2ccc(NC(=O)C=C3CCC3)cc2)CC1]

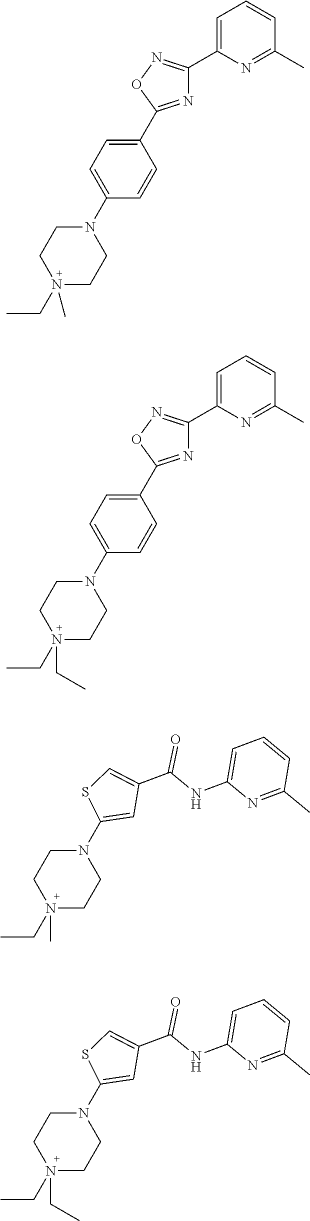

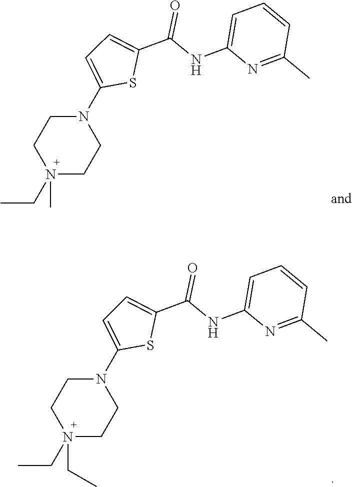

5. The compound of claim 1, wherein the compound is at least 70%, at least 80%, at least 90%, at least 95%, at least 98%, or at least 99% enantiomerically pure with respect to one or more chiral sites.

6. The compound of claim 1, wherein the compound is peripherally active and does not substantially cross the blood-brain barrier.

7. The compound of claim 1, wherein the compound is at least partially uncharged at physiological pH and is capable of crossing the blood-brain barrier.

8. The compound of claim 1, wherein the compound is present as a pharmaceutically acceptable salt, such as a halide, or a salt formed with an acid, such as a hydrochloride.

9. The compound of claim 1, wherein the compound has a dissociation constant of less than about 1 μM, or less than about 300 nm, or less than about 200 nM, or less than about 100 nM, for a form of the nicotinic acetylcholine receptor comprising one or more alpha7, alpha9, and/or alpha10 subunits.

10. The compound of claim 1, wherein the compound has a binding selectivity for alpha9-containing forms and/or alpha9-alpha10-containing forms of the nicotinic acetylcholine receptor over alpha7-containing forms of the nicotinic acetylcholine receptor of at least 50, at least 100, at least 150, at least 200, or at least 250.

11. The compound of claim 1, wherein the compound decreases pain and/or inflammation when administered to a mammal at an effective dose.

12. A pharmaceutical composition comprising the compound of claim 1 and at least one excipient.

13. The pharmaceutical composition of claim 12, further comprising one or more additional active agents.

14. The pharmaceutical composition of claim 13, wherein the one or more additional agents comprise an agent for treatment of pain, inflammation, or cancer.

15. A method to aid in treating, or preventing or alleviating to any degree, a disorder related to a nicotinic acetylcholine receptor comprising an alpha9, alpha10 and/or alpha7 subunit, the method comprising administering to a mammalian subject in need thereof an effective amount of the compound of claim 1.

16. The method of claim 15, wherein the disorder is selected from the group consisting of sensory and auditory disorders; hearing loss (including noise-induced, age-related, or ototoxic); tinnitus; pain; inflammation; neuropathic pain; chronic pain (including inflammatory, musculoskeletal, cancer-induced); visceral pain (including interstitial cystitis and irritable bowel syndrome); neurodegenerative disorders; neurological disorders; multiple sclerosis; Parkinson's disease; peripheral neuropathy; autoimmune disorders; rheumatoid arthritis; Inflammatory bowel disease (including Crohn's disease, ulcerative colitis); cancer (including cancer chemotherapy and pain related to oral, bone, or visceral cancers; chemotherapy-induced hearing loss or neuropathy; preventive care related to platinum-based or taxane-based chemotherapies; and cancer immunomodulation.

17. The method of claim 15, wherein the disorder is selected from the group consisting of pain, chronic pain, neuropathic pain, inflammation, inflammatory pain, neuroinflammation, tinnitus, and an inner ear disorder.
